SEQUENCE LISTING (25 sequences)

SEQ ID NO: 1 - protein, 575 aa, Bos taurus
MRPWTGSWRWIMLILFAWGTLLFYIGGHLVRDNDHPDHSSRELSKILAKLERLKQQNEDLRRMAESLRIPEGPIDQGPASGRIRALEEQLVKAKEQIENYKKQTRNGLGKDHEILRRRIENGAKELWFFLQSELKKLKNLEGNELQRHADEFLSDLGHHERSIMTDLYYLSQTDGAGDWREKEAKDLTELVQRRITYLQNPKDCSKAKKLVCNINKGCGYGCQLHHVVYCFMIAYGTQRTLILESHNWRYATGGWETVFRPVSETCTDRSGVYTGHWSGEIKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPHYEFISDNSISWSAGLHNRYTENSLRGVILDIHFLSQADFLVCTFSSQVCRVAYEIMQTLHPDASANFHSLDDIYYFGGQNAHNQIAIYPHEPRTADEIPMEPGDIIGVAGNHWDGYSKGVNRKLGRTGLYPSYKVREKIETVKVPHVPEAEK

SEQ ID NO: 2 - protein, 619 aa, Drosophila melanogaster
MLLVRQLFGASANSWARALIIFVLAWIGLVYVFVVKLTNTQGQQAAGESELNARRISQALQMLEHTRQRNEELKQLIDELMSDQLDKQSAMKLVQRLENDALNPKLAPEVAGPEPESMFESAPADLRGWNNVAEGAPNDLEAGVPDHGEFEPSLEYEFTRRRIQTNIGEIWNFFSSELGKVRKAVAAGHASADLEESINQVLLQGAEHKRSLLSDMERMRQSDGYEAWRHKEARDLSDLVQRRLHHLQNPSDCQNARKLVCKLNKGCGYGCQLHHVVYCFIVAYATERTLILKSRGWRYHKGGWEEVFQPVSNSCHDAGTANTYNWPGKPNTQVLVLPIIDSLMPRPPYLPLAVPEDLAPRLKRLHGDPIVWWVGQFLKYLLRPQPTTRDFLTSGMRNLGWERPIVGVHVRRTDKVGTEAACHSVEEYMTYVEDYYRTLEVNGSTVARRIFLASDDAQVIEEARRKYPQYQIIGDPEVARMASVSTRYTDTALNGIILDIHLLSMSDHLVCTFSSQVCRVAYEIMQTMYPDAAHRFKSLDDIYYYGGQNAHNRRVVIAHKPRTHEDLQLRVGDLVSVAGNHWDGNSKGKNTRTNQGGLFPSFKVEEKVDTAKLPLYAGI

SEQ ID NO: 3 - protein, 575 aa, Homo sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 4 - protein, 575 aa, Mus musculus
MRAWTGSWRWIMLILFAWGTLLFYIGGHLVRDNDHPDHSTRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEQHFQLLARRMQVDKKRVYLATDDPTLLKEANTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 5 - protein, 575 aa, Pan troglodytes; identical to SEQ ID NO: 3

SEQ ID NO: 6 - protein, 559 aa, Caenorhabditis elegans
MLKCIAAVGTVVWMTMFLFLYSQLSNNQSGGDSIRAWRQTKEAIDKLQEQNEDLKSILEKERQERNDQHKKIMEQSHQLPPNPENPSLPKPEPVKEIISKPSILGPVQQEVQKRMLDDRIREMFYLLHSQTIENSTKILLETQMISLMGLSAQLEKLEGSEEERFKQRTAITQRIFKSIEKLQNPKACSEAKTLVCNLDKECGFGCQLHHVTYCAITAFATQRMMVLKRDGSSWKYSSHGWTSVFKKLSKCSFDEAVGNTEAKPFAEPSPARVVSLGIVDSLITKPTFLPQAVPEQLLESLTSLHSHPPAFFVGTFISYLMRFNSATQEKLDKALKSIPLDKGPIVGLQIRRTDKVGTEAAFHALKEYMEWTEIWFKVEEKRQGKPLERRIFIASDDPTVVPEAKNDYPNYEVYGSTEIAKTAQLNNRYTDASLMGVITDIYILSKVNYLVCTFSSQVCRMGYELRQPSGADDGSKFHSLDDIYYFGGQQAHEVIVIEDHIAQNNKEIDLKVGDKVGIAGNHWNGYSKGTNRQTYKEGVFPSYKVVNDWRKFKFEALLD

SEQ ID NO: 7 - protein, 603 aa, Dictyostelium discoideum
MNRNSWFCCLILLIIIYLLSQIIINKISNTFAIKQPKLNNITVNYNFKNNIEIVDLIFIQYYNPLEKEYIKIINNNHSVLNYFTSNLINSHKSYKNFKIINFLNNNNNNNKSPILKYNIENNNNNNNKIFKDISFSNNKNIFDKLISNHLILEITSLNDLIDLFDFIEFIEGKIKNSITIFGFCFLRENLEENIDNFLSSHRVWSIKFNYSFNCKLNNYEKSFEYGQLIQLIKVSESFDTFKFYNYLDKLQFYNNNNGGNKNNYNNKILIHSLRPFGMISSLHFLGYSLTLSLEWNRTLIIDDSKFLFSNKFTDLFLPITAKTKFENFNNVEEKSAQIINFLKSEKEYNHSNPISIITNDYHVYVDFKSCNEFPKQWFQGGDLYCFKSHIMNYIIRPNYKIRKIIELHKLNLFHNDKNNYNINNDELNCLAIHIRNGDKVIENSMKNKSIVLNYFQDYLDFIVNDKYLNNGRNFIKNIFVMSDNQTIFNIDLPNAQIRYPQFKFHYLKDIIRDDNTTNFIKYLNDDNYDTNLKTLKNRPNSNIGNGLLSEIIIASECQYFIGSQTSNVARLIVELMNANRKSNPSLKIKLYKTLDNSSWFADP

SEQ ID NO: 8 - protein, 575 aa, Gallus gallus
MRPWTGSWRWIMLILFAWGTLLFYIGGHLVRDSEHPDHSSRELSKILAKLERLKQQNEDLRRMAESLRIPDGPIDQGPAAGKVHALEEQLLKAKEQIENYKKQTGDGLGKDHEILRRRIENGAKELWFFLQSELKKLKNLEGSELQRRIDEFLSDLGHQERSIMTDLYYLSQTDGAGDWREKEAKDLTDLVQRRITYLQNPKDCSKAKKLVCNINKGCGYGCQLHHVVYCFMIAYGTQRTLILESQNWRYATGGWETVFRPVSETCTDRSGTTTGHWSGETNDKDVQVVELPIVDSLHPRPPYLPLAVPEDLADRLIRVHGDPAVWWVSQFVKYLIRPQPWLEKEIEEATRKLGFKHPVIGVHVRRTDKVGTEAAFHPIEEYMVHVEERFELLARRMHVDKKRVYLATDDPSLLQEAKSKYPNYEFISDNSISWSAGLHNRYTENSLRGVILDIHFLSQADFLVCTFSSQVCRVPYEIMQTLHPDASAYFHSLDDIYYFGGQNAHNQIAVYAHHPRTADEIPMEPGDIIGVAGNHWDGYSKGINRKLGKTGLYPSYKVKEKIETVKYPTYPEAEK

SEQ ID NO: 9 - protein, 575 aa, Canis familiaris
MRPWTGSWRWIMLILFAWGTLLFYIGGHLVRDNDHPDHSSRELSKILAKLERLKQQNEDLRRMAESLRIPEGPIDQAPASGRVRALEEQLLKAKEQIENYKKQTRNGLGKDHEILRRRIENGAKELWFFLQSELKKLKNLEGNVLQRHADEFLSDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVNETCTDRSGTSTGHWSGEVKDKNVQVVELPIVDSLHPRPPYLPLAVPEDLADRLVRVHGDPAVWWVSQFVKYLIRPQPWLEKEIEEATKKLGFNIPVIGVHVRRTDKVGTEAAFHPIEEYMVHVEEHFQLLARRMQVDKRRVYLATDDPSLLKEAKTKYPTYEFISDNSISWSAGLHNRYTENSLRGVILDIHFLSQADFLVCTFSSQVCRVAYEIMQTLHPDASANFHSLDDIYYFGGQNAHNQIAIYPHQPRTADEIPMEPGDIIGVAGNHWDGYSKGVNRKLGRTGLYPSYKVREKIETVKYPTYPEAEK

SEQ ID NO: 10 - protein, 575 aa, Rattus norvegicus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHELLRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPALLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTDEEIPMEPGDIIGVAGNHWDGYSKGVNRKLGKTGLYPSYKVREKIETVKYPTYPEAEK

SEQ ID NO: 11 - protein, 575 aa, Sus scrofa
MRPWTGSWRWIMLILFAWGTLLFYIGGHLVRDNDHSDHSSRELSKILAKLERLKQQNEDLRRMAESLRIPEGPIDQGPASGRVRALEEQFMKAKEQIENYKKQTKNGPGKDHEILRRRIENGAKELWFFLQSELKKLKNLEGNELQRHADEFLSDLGHHERSIMTDLYYLSQTDGAGDWREKEAKDLTELVQRRITYLQNPKDCSKAKKLVCNINKGCGYGCQLHHVVYCFMIAYGTQRTLALESHNWRYATGGWETVFRPVSETCTDRSGSSTGHWSGEVKDKNVQVVELPIVDSVHPRPPYLPLAVPEDLADRLVRVHGDPAVWWVSQFVKYLIRPQPWLEKEIEEATKKLGFKHPVIGVHVRRTDKVGAEAAFHPIEEYTVHVEEDFQLLARRMQVDKKRVYLATDDPALLKEAKTKYPSYEFISDNSISWSAGLHNRYTENSLRGVILDIHFLSQADFLVCTFSSQVCRVAYEIMQALHPDASANFRSLDDIYYFGGPNAHNQIAIYPHQPRTEGEIPMEPGDIIGVAGNHWDGYPKGVNRKLGRTGLYPSYKVREKIETVKYPTYPEADK

SEQ ID NO: 12 - DNA, 830 bp, Macaca mulatta
aaacggtcaagtaccccacatatcctgaggctgagaaataaagctcagatggaagagataaacaaccaaactcagttcaaaccatttgagccaaactgtagatgaagagggctctgatctaacaaaataaggttatatgagtagataccctcagcaccaagagcagctgggaactgacagaggcttcaattggtggaattcctctttaacaagggctgcaatgccctcaaacccatgcacagtacaataatgtactcacatataacatgcaaacaggttttctactttgtccctttctttcaatgtgtccccataagacaaacactgccatattgtgtaatttaagtgacacagacattttgtgtgagacttaaaacatggtgcctatatctgagagacctatgtgaactattgagaagacctgaacagctccttactctgacgaagttgattcttatttggtggtggtattgtgaccactgcattcactccggtcaacagattcagaatgagaatggacgtttggtttttgtttttgtttttgtttttttcctttataaggttatctgggttttttttttttaaataattgcatcagttcattgacctcatcattaataagtgaagaatacatcggaaaataaaatattcactctccattagaaaattttgtaaaacaatgccatgaacaaattctttagtactcaatgtttctggacatactctttgataacaaaaataaattttaaaaaggaattttgtaaagtttctagaactttgtatcattggatgttatgatgatcagcctcatgtggaagaactgtgataaa

SEQ ID NO: 13 - DNA, 2008 bp, Cricetulus griseus
aacagaaacttattttcctgtgtggctaactagaaccagagtacaatgtttccaattctttgagctccgagaagacagaagggagttgaaactctgaaaatgcgggcatggactggttcctggcgttggattatgctcattctttttgcctgggggaccttattgttttatataggtggtcatttggttcgagataatgaccaccctgaccattctagcagagaactctccaagattcttgcaaagctggagcgcttaaaacaacaaaatgaagacttgaggagaatggctgagtctctccgaataccagaaggccctattgatcaggggacagctacaggaagagtccgtgttttagaagaacagcttgttaaggccaaagaacagattgaaaattacaagaaacaagctaggaatgatctgggaaaggatcatgaaatcttaaggaggaggattgaaaatggagctaaagagctctggttttttctacaaagtgaattgaagaaattaaagaaattagaaggaaacgaactccaaagacatgcagatgaaattcttttggatttaggacatcatgaaaggtctatcatgacagatctatactacctcagtcaaacagatggagcaggtgagtggcgggaaaaagaagccaaagatctgacagagctggtccagcggagaataacatatctgcagaatcccaaggactgcagcaaagccagaaagctggtatgtaatatcaacaaaggctgtggctatggatgtcaactccatcatgtggtttactgcttcatgattgcttatggcacccagcgaacactcatcttggaatctcagaattggcgctatgctactggaggatgggagactgtgtttagacctgtaagtgagacatgcacagacaggtctggcctctccactggacactggtcaggtgaagtgaaggacaaaaatgttcaagtggtcgagctccccattgtagacagcctccatcctcgtcctccttacttacccttggctgtaccagaagaccttgcagatcgactcctgagagtccatggtgatcctgcagtgtggtgggtatcccagtttgtcaaatacttgatccgtccacaaccttggctggaaagggaaatagaagaaaccaccaagaagcttggcttcaaacatccagttattggagtccatgtcagacgcactgacaaagtgggaacagaagcagccttccatcccattgaggaatacatggtacacgttgaagaacattttcagcttctcgaacgcagaatgaaagtggataaaaaaagagtgtatctggccactgatgacccttctttgttaaaggaggcaaagacaaagtactccaattatgaatttattagtgataactctatttcttggtcagctggactacacaaccgatacacagaaaattcacttcggggcgtgatcctggatatacactttctctcccaggctgacttccttgtgtgtactttttcatcccaggtctgtagggttgcttatgaaatcatgcaaacactgcatcctgatgcctctgcaaacttccattctttagatgacatctactattttggaggccaaaatgcccacaaccagattgcagtttatcctcaccaacctcgaactaaagaggaaatccccatggaacctggagatatcattggtgtggctggaaaccattggaatggttactctaaaggtgtcaacagaaaactaggaaaaacaggcctgtacccttcctacaaagtccgagagaagatagaaacagtcaaataccctacatatcctgaagctgaaaaatagagatggagtgtaagagattaacaacagaatttagttcagaccatctcagccaagcagaagacccagactaacatatggttcattgacagacatgctccgcaccaagagcaagtgggaaccctcagatgctgcactggtggaacgcctctttgtgaagggctgctgtgccctcaagcccatg

SEQ ID NO: 14 - DNA, 19 bp, Artificial Sequence
  Description of Artificial Sequence Synthetic oligonucleotide
ccagaaggccctattgatc

SEQ ID NO: 15 - DNA, 20 bp, Artificial Sequence
  Description of Artificial Sequence Synthetic oligonucleotide
gccagaaggccctattgatc

SEQ ID NO: 16 - DNA, 21 bp, Artificial Sequence
  Description of Artificial Sequence Synthetic oligonucleotide
gatcaatagggccttctggta

SEQ ID NO: 17 - DNA, 9 bp, Artificial Sequence
  Description of Artificial Sequence Synthetic oligonucleotide
ttcaagaga

SEQ ID NO: 18 - DNA, 28 bp, Artificial Sequence
  Description of Artificial Sequence Synthetic oligonucleotide
tcgagggcgcgccagctcgggccggcca

SEQ ID NO: 19 - DNA, 28 bp, Artificial Sequence
  Description of Artificial Sequence Synthetic oligonucleotide
agcttggccggcccgagctggcgcgccc

SEQ ID NO: 20 - DNA, 66 bp, Artificial Sequence
  Description of Artificial Sequence Synthetic oligonucleotide
gatccgccagaaggccctattgatcttcaagagagatcaatagggccttctggtattttttggaaa

SEQ ID NO: 21 - DNA, 66 bp, Artificial Sequence
  Description of Artificial Sequence Synthetic oligonucleotide
agcttttccaaaaaataccagaaggccctattgatctctcttgaagatcaatagggccttctggcg

SEQ ID NO: 22 - DNA, 20 bp, Artificial Sequence
  Description of Artificial Sequence Synthetic primer
ggcgttggattatgctcatt

SEQ ID NO: 23 - DNA, 20 bp, Artificial Sequence
  Description of Artificial Sequence Synthetic primer
ccctgatcaatagggccttc

SEQ ID NO: 24 - DNA, 20 bp, Artificial Sequence
  Description of Artificial Sequence Synthetic primer
ccgatgctgctaagaacaca

SEQ ID NO: 25 - DNA, 20 bp, Artificial Sequence
  Description of Artificial Sequence Synthetic primer
cttcagttccagcccaactc

(19) United States
(12) Patent Application Publication
(10) Pub. No.: US 2010/0015627 A1
(43) Pub. Date: Jan. 21, 2010

(54) SELECTION METHOD

(76) Inventors: Vincent Beuger, Koeln (DE); Helmut Burtscher, Habach (DE); Christian Klein, Iffeldorf (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/518,112

(22) PCT Filed: Dec. 19, 2007
(86) PCT No.: PCT/EP2007/011158

§ 371 Date: Jun. 8, 2009

(30) Foreign Application Priority Data
Dec. 22, 2006 (EP) 06026654.1

(51) Int. Cl. C12Q 1/68; C07H 21/04

(52) U.S. Cl. 435/6; 536/23.1

(57) ABSTRACT

The current invention comprises a method for the selection of a mammalian cell by transfecting a mammalian cell with a nucleic acid comprising a part of a nucleic acid encoding a polypeptide that catalyzes an α1,6-glycosidic bond formation between fucose and an asparagine-linked N-acetylglucosamine and cultivating the transfected mammalian cell in the presence of Lens culinaris agglutinin (LCA) and selecting a mammalian cell viable under these conditions.

The present invention relates to the field of RNAi. More precisely, the present invention relates to the field of reducing the translation of polypeptides in cells and subsequent selection of said cells.

### BACKGROUND OF THE INVENTION

The phenomenon of RNAi mediated gene silencing has been described first in the Caenorhabditis elegans system, in which microinjection of long double stranded RNA molecules was reported to result in an inactivation of the respective gene (U.S. Pat. No. 6,506,559). Later on, RNAi mediated gene silencing has been disclosed in vertebrates (EP 1 114 784), in mammals, and in particular in human cells (EP 1 144 623). In these systems, gene inactivation is achieved successfully, if short, double stranded RNA molecules of 19-29 bp are transfected in order to transiently knock down a specific gene of interest.

The mechanism of RNA mediated gene inactivation seems to be slightly different in the various organisms that have been investigated so far. In all systems, however, RNA mediated gene silencing is based on post-transcriptional degradation of the target mRNA induced by the endonuclease Argonaute2 which is part of the so called RISC complex (WO 03/93430). Sequence specificity of degradation is determined by the nucleotide sequence of the specific antisense RNA strand loaded into the RISC complex.

Appropriate possibilities of introduction include transfecting the double stranded RNA molecule itself, or in vivo expression of DNA vector constructs, which directly result in short double stranded RNA compounds having a sequence that is identical or complementary to a part of the target RNA molecule. In many cases, so called shRNA constructs have been used successfully for gene silencing. These constructs encode a stem-loop RNA, characterized in that after introduction into cells, it is processed into a double stranded RNA compound, the sequence of which corresponds to the stem of the original RNA molecule.

IgG1-type immunoglobulins have two N-linked oligosaccharide chains bound to the Fc region at position Asn297 or in some cases at position Asn298. N-linked oligosaccharides generally are of the complex biantennary type, composed of a trimannose core structure with presence or absence of core fucose (Rademacher, T. W., et al., Biochem. Soc. Symp. 51 (1986) 131-148; Umana, P., et al., Nature Biotechnol. 17 (1999) 176-180; Okazaki, A., et al., J. Mol. Biol. 336 (2004) 1239-1249; Shinkawa, T., et al., J. Biol. Chem. 278 (2003) 3466-3473).

Longmore and Schachter (Longmore, G. D. and Schachter, H., Carbohydrate Res. 100 (1982) 365-392) isolated from porcine liver the enzyme α1,6-fucosyltransferase (FuT8). FuT8 is located in the glycosylation pathway of cells and catalyzes the fucosylation of the innermost N-acetylglucosamine residue of N-linked oligosaccharides. The linkage is a α1,6-glycosidic bond.

US 2004/0132140 and US 2004/0110704 report recombinant and/or genetic methods for inhibiting α1,6-fucosyltransferase within cell lines expressing recombinant immunoglobulins.

LCA (Lens culinaris agglutinin) is a lectin that recognizes α1,6-fucosylated trimannose-core structures of N-linked oligosaccharides. Cells presenting fucose structures on their cell surface are recognized and lysed by LCA (Ripka, J. and Stanley, P., Som. Cell Mol. Gen. 12 (1986) 51-62; Mori, K., et al., Biotechnol. Bioeng. 88 (2004) 901-908). In EP 1 705 251 is a process for producing antibody compositions by using RNA inhibiting reported.

### SUMMARY OF THE INVENTION

The current invention comprises a method for selecting a mammalian cell, wherein the method comprises the following steps
a) transfecting a mammalian host cell with a nucleic acid that comprises a first nucleic acid comprising SEQ ID NO: 14, 15, or 16, which contains a part of a nucleic acid encoding a polypeptide that catalyzes an α1,6-glycosidic bond formation between fucose and an asparagine-linked N-acetylglucosamine,
b) cultivating the transfected mammalian cell in the presence of Lens culinaris agglutinin, and
c) selecting a mammalian cell viable under the conditions of step b).

In one embodiment the first nucleic acid is transcribed to a short hairpin nucleic acid (shRNA).

The nucleic acid according to the method of the invention comprises in one embodiment a second nucleic acid encoding a selection marker. In an other embodiment the nucleic acid comprises a third nucleic acid encoding a heterologous polypeptide. Preferably said heterologous polypeptide is an immunoglobulin, an immunoglobulin fragment, or an immunoglobulin conjugate. In one embodiment the first nucleic acid comprises a nucleic acid selected from the group comprising SEQ ID NO: 14 to SEQ ID NO: 16. In another embodiment is the first nucleic acid the nucleic acid of SEQ ID NO: 20, or SEQ ID NO: 21. The invention comprises a method for selecting a mammalian cell, wherein the method comprises the following steps
a) transfecting a mammalian host cell with a nucleic acid that comprises a first nucleic acid of SEQ ID NO: 20, or 21, which contains a part of a nucleic acid encoding a polypeptide that catalyzes an α1,6-glycosidic bond formation between fucose and an asparagine-linked N-acetylglucosamine,
b) cultivating the transfected mammalian cell in the presence of Lens culinaris agglutinin, and
c) selecting a mammalian cell viable under the conditions of step b).

The nucleic acid according to this method of the invention comprises in one embodiment a second nucleic acid encoding a selection marker. In an other embodiment the nucleic acid comprises a third nucleic acid encoding a heterologous polypeptide. Preferably said heterologous polypeptide is an immunoglobulin, an immunoglobulin fragment, or an immunoglobulin conjugate.

The method according to the invention in a further embodiment comprises between step a) and step b) two additional steps
a1) cultivating said transfected mammalian cell in the presence of a selection agent,
a2) selecting a mammalian cell viable under the conditions of step a1).

In one embodiment of the method according to the invention the cultivating of the transfected mammalian cell is with an increasing concentration of said selection agent and/or with an increasing concentration of LCA.

In one embodiment the mammalian cell is selected from the group of cells comprising hybridoma and rodent cells, preferably comprising CHO cells, BHK cells, Sp2/0 cells, and NS0 cells.

### DETAILED DESCRIPTION OF THE INVENTION

The current invention provides a method for the selection of a mammalian cell that can be cultivated in the presence of LCA, wherein the translation of an mRNA encoding a polypeptide that catalyzes an α1,6-glycosidic bond formation between fucose and an asparagine-linked N-acetylglucosamine, preferably encoding α1,6-fucosyltransferase, in said mammalian cell is reduced by RNAi.

Methods and techniques known to a person skilled in the art, which are useful for carrying out the current invention, are described e.g. in Ausubel, F. M., ed., Current Protocols in Molecular Biology, Volumes I to III (1997); Sambrook et al., Molecular Cloning: A Laboratory Manual, Second Edition, Cold Spring Harbor Laboratory Press, Cold Spring Harbor, N.Y. (1989); Glover, D. M., and Hames, B. D., ed., DNA Cloning: A Practical Approach, Volumes I and II (1995), Oxford University Press; Freshney, R. I. (ed.), Animal Cell Culture—a practical approach, IRL Press (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Techniques, Second Edition, Alan R. Liss, Inc., N.Y. (1987).

The use of recombinant DNA technology enables the production of numerous derivatives of a nucleic acid and/or polypeptide. Such derivatives can, for example, be modified in one individual or several positions by substitution, alteration, exchange, deletion, or insertion. The modification or derivatisation can, for example, be carried out by means of site directed mutagenesis. Such modifications can easily be carried out by a person skilled in the art (see e.g. Sambrook, J., et al., Molecular Cloning: A laboratory manual (1989) Cold Spring Harbor Laboratory Press, New York, USA; Hames, B. D., and Higgins, S. G., Nucleic acid hybridization—a practical approach (1985) IRL Press, Oxford, England).

The use of recombinant technology enables the transformation of various host cells with heterologous nucleic acid(s). Although the transcription and translation, i.e. expression, machinery of different cells use the same elements, cells belonging to different species may have, e.g., a different so-called codon usage. Thereby identical polypeptides (with respect to amino acid sequence) may be encoded by different nucleic acid(s). Also, due to the degeneracy of the genetic code, different nucleic acids may encode the same polypeptide.

“A polypeptide that catalyzes an α1,6-glycosidic bond formation between fucose and an asparagine-linked N-acetylglucosamine” is a catalytically active polypeptide, i.e. an enzyme, which is able to form an α-glycosidic bond between the 1-position of fucose and the 6-position of N-acetylglucosamine. The N-acetylglucosamine itself is preferably N-glycosidically bound to the nitrogen of the free amide group (H₂N—CO—) of the amino acid asparagine, preferably of asparagine-297 or asparagine-298 of an immunoglobulin heavy chain (numbering according to Kabat, see e.g. Johnson, G. and Wu, T. T., Nucleic Acids Res. 28 (2000) 214-218). The N-acetylglucosamine is preferably located at one end of a sugar chain, i.e. is the terminal sugar residue of an oligosaccharide.

A “nucleic acid” as used herein, refers to a polynucleotide molecule, for example to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed, e.g. by mutagenesis, deleted, or added. A nucleic acid can either be isolated, or integrated in another nucleic acid, e.g. in an expression plasmid, or the chromosome of a host cell. A nucleic acid is likewise characterized by its nucleic acid sequence consisting of individual nucleotides.

To a person skilled in the art procedures and methods are well known to convert an amino acid sequence of, e.g., a polypeptide into a corresponding nucleic acid sequence encoding the amino acid sequence. Therefore, a nucleic acid is characterized by its nucleic acid sequence consisting of individual nucleotides and likewise by the amino acid sequence of a polypeptide encoded thereby.

The expression “a part of a nucleic acid encoding a polypeptide” denotes a nucleic acid which is a partial nucleic acid, i.e. a fraction of a complete nucleic acid, preferably of an mRNA. A complete nucleic acid is e.g. a structural gene. The complete nucleic acid comprises all transcribed nucleotides of the corresponding gene. The part of a nucleic acid or the partial nucleic acid comprises any consecutive fraction of the complete nucleic acid having a length of from 5 to 55 nucleotides, preferably of 10 to 40 nucleotides, preferably of 19 to 29 nucleotides, more preferably of 19 to 23 nucleotides.

The expression “plasmid” includes e.g. shuttle and expression vectors/plasmids as well as transfection vectors/plasmids. The terms “plasmid” and “vector” are used interchangeably within this application. Typically, a “plasmid” will also comprise an origin of replication (e.g. the ColE1 or oriP origin of replication) and a selection marker (e.g. an ampicillin, kanamycin, tetracycline, or chloramphenicol selection marker), for replication and selection, respectively, of the vector/plasmid in bacteria.

An “expression cassette” refers to a construct that contains the necessary regulatory elements, such as promoter and polyadenylation site, for expression of at least the contained nucleic acid, e.g. of a structural gene, in a host cell. Optionally additional elements may be contained which e.g. enable the secretion of the expressed polypeptide. The term “expression cassette” is also applicable if the nucleic acid contained in the expression cassette is only transcribed but not translated, as e.g. in case of a shRNA.

A “structural gene” denotes the coding region of a gene without a signal sequence.

A “gene” denotes a nucleic acid segment, e.g. on a chromosome or on a plasmid, which is necessary for the expression of a polypeptide or protein. Beside the coding region the gene comprises other functional elements including promoters, introns, terminators, and optionally a leader peptide.

A “selection marker” is a nucleic acid that allows cells carrying the selection marker to be specifically selected for or against, in the presence of a corresponding selection agent. A useful positive selection marker is an antibiotic resistance gene. This selection marker allows the host cell transformed therewith to be positively selected for in the presence of the corresponding selection agent, e.g. the antibiotic. A non-transformed host cell is not capable to grow or survive under the selective conditions in the culture. A selection marker can be positive, negative, or bifunctional. Positive selection markers allow selection for cells carrying the marker, whereas negative selection markers allow cells carrying the marker to be selectively eliminated. Typically, a selection marker will confer resistance to a drug or compensate for a metabolic or catabolic defect in the host cell. Selection markers used with eukaryotic cells include, e.g., the genes for aminoglycoside phosphotransferase (APH), such as e.g. the hygromycin (hyg), neomycin (neo), and G418 selection marker, dihydrofolate reductase (DHFR), thymidine kinase (tk), glutamine synthetase (GS), asparagine synthetase, tryptophan synthetase (selection agent indole), histidinol dehydrogenase (selection agent histidinol D), and nucleic acids conferring resistance to puromycin, bleomycin, phleomycin, chloramphenicol, Zeocin, and mycophenolic acid. Further marker genes are described e.g. in WO 92/08796 and WO 94/28143.

The term “expression” as used herein refers to transcription and/or translation processes occurring within a cell. In case of a RNAi compound refers “expression” to transcription and in case of a nucleic acid encoding a heterologous polypeptide to transcription and translation. The level of transcription of a desired product in a host cell can be determined on the basis of the amount of corresponding mRNA that is present in the cell. For example, mRNA transcribed from a sequence of interest can be quantitated by PCR or by Northern hybridization (see Sambrook et al., Molecular Cloning: A Laboratory Manual, Cold Spring Harbor Laboratory Press (1989)). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook et al., 1989, supra).

A “host cell” refers to a cell into which a nucleic acid, e.g. encoding a heterologous polypeptide or constituting a shRNA, can be introduced. Host cells include both prokaryotic cells, which are used for propagation of vectors/plasmids, and eukaryotic cells, which are used for the expression of nucleic acids. Preferably, the eukaryotic cells are mammalian cells. Preferably the mammalian host cell is selected from the group of mammalian cells comprising CHO cells (e.g. CHO K1 or CHO DG44), BHK cells, NS0 cells, SP2/0 cells, HEK 293 cells, HEK 293 EBNA cells, PER.C6® cells, and COS cells. Preferably the mammalian cell is selected from the group comprising hybridoma, myeloma, and rodent cells. Myeloma cells comprise rat myeloma cells (e.g. YB2), and mouse myeloma cells (e.g. NS0, SP2/0). In one embodiment said host cell is a CHO cell.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is produced, and may vary with the type of cell. Polypeptides are defined herein in terms of their amino acid backbone structure. Additions such as carbohydrate groups are generally not specified, but may be present nonetheless.

The term “amino acid” as used within this application denotes a group of carboxy α-amino acids, which directly or in form of a precursor can be encoded by a nucleic acid, comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

As used herein, the term “immunoglobulin” denotes a protein consisting of one or more polypeptides substantially encoded by immunoglobulin genes. This definition includes variants such as mutated forms, i.e. forms with substitutions, deletions, and insertions of one or more amino acids, truncated forms, fused forms, chimeric forms, as well as humanized forms. The recognized immunoglobulin genes include the different constant region genes as well as the myriad immunoglobulin variable region genes from, e.g., primates and rodents. Immunoglobulins may exist in a variety of formats, including, for example, Fv, Fab, and (Fab)₂, as well as single chains (scFv) (e.g. Huston, J. S., et al., Proc. Natl. Acad. Sci. USA 85 (1988) 5879-5883; Bird, R. E., et al., Science 242 (1988) 423-426; and, in general, Hood et al., Immunology, Benjamin N.Y., 2nd edition (1984) and Hunkapiller, T., and Hood, L., Nature 323 (1986) 15-16). Monoclonal immunoglobulins are preferred.

Each of the heavy and light polypeptide chains of an immunoglobulin, if present at all, may comprise a constant region (generally the carboxyl terminal portion). The constant region of the heavy chain mediates the binding of the immunoglobulin i) to cells bearing an Fc-gamma receptor (FcγR), such as phagocytic cells, or ii) to cells bearing the neonatal Fc receptor (FcRn) also known as Brambell receptor. It also mediates the binding to some factors including factors of the classical complement system such as component (C1q).

Each of the heavy and light polypeptide chains of an immunoglobulin, if present at all, may comprise a variable domain (generally the amino terminal portion). The variable domain of an immunoglobulin's light or heavy chain may comprise different regions, i.e. four framework regions (FR) and three hypervariable regions (CDR).

The term “monoclonal immunoglobulin” as used herein refers to an immunoglobulin obtained from a population of substantially homogeneous immunoglobulins, i.e. the individual immunoglobulins comprising the population are identical except for possible naturally occurring mutations that may be present in minor amounts. Monoclonal immunoglobulins are highly specific, being directed against a single antigenic site. Furthermore, in contrast to polyclonal immunoglobulin preparations, which include different immunoglobulins directed against different antigenic sites (determinants or epitopes), each monoclonal immunoglobulin is directed against a single antigenic site on the antigen. In addition to their specificity, the monoclonal immunoglobulins are advantageous in that they may be synthesized uncontaminated by other immunoglobulins. The modifier “monoclonal” indicates the character of the immunoglobulin as being obtained from a substantially homogeneous population of immunoglobulins and is not to be construed as requiring production of the immunoglobulin by any particular method.

“Humanized” forms of non-human (e.g. rodent) immunoglobulins are chimeric immunoglobulins that contain partial sequences derived from non-human immunoglobulin and from human immunoglobulin. For the most part, humanized immunoglobulins are derived from a human immunoglobulin (recipient immunoglobulin), in which residues from a hypervariable region are replaced by residues from a hypervariable region of a non-human species (donor immunoglobulin), such as mouse, rat, rabbit, or non-human primate, having the desired specificity and affinity (see e.g. Morrison, S. L., et al., Proc. Natal. Acad. Sci. USA 81 (1984) 6851-6855; U.S. Pat. No. 5,202,238; U.S. Pat. No. 5,204,244). In some instances, framework region (FR) residues of the human immunoglobulin are replaced by corresponding non-human residues. Furthermore, humanized immunoglobulin may comprise further modifications, e.g. amino acid residues that are not found in the recipient immunoglobulin or in the donor immunoglobulin. Such modifications result in variants of such recipient or donor immunoglobulin, which are homologous but not identical to the corresponding parent sequence. These modifications are made to further refine immunoglobulin performance.

In general, the humanized immunoglobulin will comprise substantially all of at least one, and typically two, variable domains, in which all or substantially all of the hypervariable loops correspond to those of a non-human donor immunoglobulin and all or substantially all of the FRs are those of a human recipient immunoglobulin. The humanized immunoglobulin optionally will also comprise at least a portion of an immunoglobulin constant region, typically that of a human immunoglobulin.

Methods for humanizing non-human immunoglobulin have been described in the art. Preferably, a humanized immunoglobulin has one or more amino acid residues introduced into it from a source which is non-human. These non-human amino acid residues are often referred to as “import” residues, which are typically taken from an “import” variable domain. Humanization can be essentially performed following the method of Winter and co-workers (Jones, P. T., et al., Nature 321 (1986) 522-525; Riechmann, L., et al., Nature 332 (1988) 323-327; Verhoeyen, M., et al., Science 239 (1988) 1534-1536; Presta, L. G., Curr. Op. Struct. Biol. 2 (1992) 593-596), by substituting hypervariable region sequences for the corresponding sequences of a human immunoglobulin. Accordingly, such “humanized” immunoglobulins are chimeric immunoglobulins (see e.g. U.S. Pat. No. 4,816,567), wherein substantially less than an intact human variable domain has been substituted by the corresponding sequence from a non-human species. In practice, humanized immunoglobulins are typically human immunoglobulins in which some hypervariable region residues and possibly some framework region residues are substituted by residues from analogous sites in rodent or non-human primate immunoglobulins.

Recombinant production of immunoglobulins is well-known in the state of the art and reported, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-160; and Werner, R. G., Drug Res. 48 (1998) 870-880.

Preferably the third nucleic acid in the method according to the invention encodes a heterologous polypeptide. Preferably the heterologous polypeptide is selected from immunoglobulins, immunoglobulin fragments, or immunoglobulin conjugates. Preferably said immunoglobulin, immunoglobulin fragment, or immunoglobulin conjugate is a monoclonal immunoglobulin, a monoclonal immunoglobulin fragment, or a monoclonal immunoglobulin conjugate.

As used herein the term “immunoglobulin fragment” denotes a part of an immunoglobulin. Immunoglobulin fragments comprise Fv, Fab, (Fab)₂, single chains (scFv), as well as single heavy chains and single light chains, as well as immunoglobulins in which at least one region and/or domain selected from the group comprising framework region 1, framework region 2, framework region 3, framework region 4, hypervariable region 1, hypervariable region 2, hypervariable region 3, each of a light and a heavy chain, Fab-region, hinge-region, variable region, heavy chain constant domain 1, heavy chain constant domain 2, heavy chain constant domain 3, heavy chain constant domain 4, and light chain constant domain, has been deleted.

As used herein the term “immunoglobulin conjugate” denotes a fusion of an immunoglobulin and a polypeptide. The term immunoglobulin conjugate comprises fusion proteins of an immunoglobulin or an immunoglobulin fragment with one to eight polypeptides, preferably with two or four polypeptides, whereby each of the polypeptides is fused to a different N- or C-terminal amino acid by an amide bond with or without an intervening linker polypeptide. If the immunoglobulin conjugate comprises more than one non-immunoglobulin polypeptide, each of the conjugated non-immunoglobulin polypeptides can have the same or a different amino acid sequence and/or length.

As used herein, the expression “cell” includes the subject cell and its progeny. Thus, the words “transformant” and “transformed cell” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

The present invention is applicable in general in all living cells expressing the so-called double-strand RNA nuclease Dicer and the RISC complex or, in other words, in all cells where RNA mediated gene silencing can be observed. Thus, the present invention is applied predominantly for mammalian cells, but can also be applied for all types of eukaryotic cells. Preferred however, are cells such as for example Chinese Hamster Ovary cells, as e.g. CHO K1 (Jones, C., et al., Cytogenet. Cell Genet. 16 (1976) 387-390), or CHO DG44 (Urlaub, G, et al., Cell 33 (1983) 405-412; Urlaub, G., et al., Somat Cell Mol Genet. 12 (1986) 555-566), Human Embryonic Kidney cells, as e.g. HEK 239 cells (Graham, F. L., et al., J. Gen. Virol. 36 (1977) 59-74), or HEK 239 EBNA cells, hybridoma cells, as e.g. NS0 cells (Barnes L. M., et al., Cytotechnology 32 (2000) 109-123; Barnes, L. M. et al., Biotech. Bioeng. 73 (2001) 261-270), or SP2/0 cells (Shulman, M., et al., Nature 276 (1978) 269-270), or Baby Hamster Kidney cells, as e.g. BHK 21 cells.

The term “reduction of translation of a polypeptide” denotes the degradation of a specific target mRNA encoding the polypeptide which is mediated by an RNAi compound. The RNAi compound itself is synthesized after transfection with an appropriate expression cassette for transcription of said RNAi compound, or after transfection with a precursor of an RNAi compound which is subsequently processed into an active RNAi compound by endogenous cellular nucleases.

Two major gene RNAi silencing strategies have emerged for in vitro studies: small interfering RNAs (siRNA) and small hairpin RNAs (shRNA) (Tuschl, T., Nat. Biotechnol. 20 (2002) 446-448).

The nucleic acid of the method according to the invention comprises a first nucleic acid, which comprises a part of a nucleic acid encoding a polypeptide that catalyzes an α1,6-glycosidic bond formation between fucose and an asparagine-linked N-acetylglucosamine. Preferably said first nucleic acid comprises SEQ ID NO: 14, 15, or 16. More preferably is the first nucleic acid of SEQ ID NO: 20, or SEQ ID NO: 21. Preferably the asparagine-linked N-acetylglucosamine is of a polysaccharide. Preferably said first nucleic acid is transcribed to an RNAi compound, more preferably to a shRNA.

The RNAi compound according to the present invention is a nucleic acid directed against the mRNA encoding a polypeptide or protein that catalyzes an α1,6-glycosidic bond formation between fucose and an asparagine-linked N-acetylglucosamine. Preferably the polypeptide is α1,6-fucosyltransferase. The transfection of cells with an RNAi compound results in cells having a reduced level of said mRNA and, thus, of the corresponding polypeptide and concurrently of the corresponding enzymatic activity. The mRNA level is of from 5% to 20%, preferably of from 5% to 15%, more preferably of from 5% to 10% of the level of the corresponding wild type cell. The wild type cell is the cell prior to the introduction/transfection of the nucleic acid of the RNAi compound.

The transcript derived from the expression cassette, which is constituting the RNAi compound, can be either transcribed from Pol II promoters such as the CMV promoter or from a Pol III promoter like the H1, U6, or 7SK promoter (Zhou, H., et al., Nucleic Acids Res. 33 (2005) e62; Brummelkamp, T. R., and Bernards, R., Nat. Rev. Cancer 3 (2003) 781-789; Czauderna, F., et al., Nucleic Acids Res. 31 (2003) e127). In case of a Pol III mediated transcription, it is essential to have a Pol III terminator sequence of TTTT, preferably a TTTTTT, at the 3′ end of the transcribed RNA for appropriate 3′ processing of the precursor RNA product (Dykxhoorn, D., et al., Nat. Rev. Mol. Cell Biol. 4 (2003) 457-467).

The RNAi compound according to the invention is preferably an RNA with a hairpin conformation, i.e. a shRNA. As an active RNAi compound, such a molecule may start with a G nucleotide at its 5′end. This is due to the fact that transcription from the H1 and U6 promoter usually starts with a G. The stem of the molecule is due to inverted repeat sequences. These are each 19 to 29, preferably 19 to 23 nucleotides in length. Preferably, these inverted repeat sequences are completely complementary to each other and can form a double stranded hybrid without any internal mismatches. One of the inverted repeat sequences is a part of a nucleic acid encoding a polypeptide that catalyzes an α1,6-glycosidic bond formation between fucose and an asparagine-linked N-acetylglucosamine. The term “asparagine-liked N-acetylglucosamine” denotes an N-acetylglucosamine that is N-glycosidically bound via its 1-position to the nitrogen of the gamma carboxyl amid group of an asparagine amino acid, whereby said asparagine amino acid is contained in a polypeptide or protein.

The internal loop of the molecule is a single stranded chain of 4 to 40 nucleotides, preferably of 4 to 9 nucleotides. For this loop, it is important to avoid any inverted repeat sequences in order to prevent the molecule from folding itself into an alternate secondary structure that is not capable of acting as a shRNA molecule.

At the 3′ end of the shRNA, there may be an overhang. In case of use of a Pol III promoter, the overhang may be 2 to 4 U residues due to the terminator signal of Pol III promoters. When expressed within a cell, these hairpin constructs are rapidly processed into active double stranded molecules capable of mediating gene silencing (Dykxhoorn, D., et al., Nat. Rev. Mol. Cell Biol. 4 (2003) 457-467).

A variety of proteins with α1,6-fucosyltransferase activity and genes encoding these from different organisms have been identified.

TABLE 1
Proteins with α1,6-fucosyltransferase activity
and encoding genes from different organisms.

| Organism | SEQ ID NO: | length |
|---|---|---|
| Bos Taurus | 01 | 575 aa |
| Drosophila melanogaster | 02 | 619 aa |
| Homo sapiens | 03 | 575 aa |
| Mus musculus | 04 | 575 aa |
| Pan troglodytes | 05 | 575 aa |
| Caenorhabditis elegans | 06 | 559 aa |
| Dictyostelium discoideum | 07 | 603 aa |
| Gallus gallus | 08 | 575 aa |
| Canis familiaris | 09 | 575 aa |
| Rattus norvegicus | 10 | 575 aa |
| Sus scrofa | 11 | 575 aa |
| Macaca mulatta | 12 | 830 nt/277 aa |
| Cricetulus griseus | 13 | 2008 nt/669 aa |

aa = amino acids; nt = nucleotides

The inverted repeat sequences of an RNAi compound (=stem) according to the invention can comprise any fraction of the translated nucleic acid encoded by the genes corresponding to the proteins of Table 1. In one embodiment the part of a nucleic acid encoding a polypeptide is a part of a nucleic acid selected from the group of nucleic acids comprising the nucleic acid sequences corresponding to the amino acid sequences of SEQ ID NO: 01 to SEQ ID NO: 11 and the nucleic acid sequences SEQ ID NO: 12 and 13.

The first nucleic acid in one embodiment comprises as the part of a nucleic acid a nucleic acid selected from the group of nucleic acids comprising the following sequences:

| | |
|---|---|
| CCAGAAGGCCCTATTGATC | (SEQ ID NO: 14) |
| GCCAGAAGGCCCTATTGATC | (SEQ ID NO: 15) |
| GATCAATAGGGCCTTCTGGTA, | (SEQ ID NO: 16) |

i.e. the first nucleic acid comprises either SEQ ID NO: 14, or SEQ ID NO: 15, or SEQ ID NO: 16.

Nucleic acids according to SEQ ID NO: 14 to 16 are denoted as stem of a shRNA. It has now surprisingly been found that with a first nucleic acid comprising a nucleic acid of SEQ ID NO: 14, 15, or 16, a reduction of the enzymatic activity of proteins with α1,6-fucosyltransferase activity in cells comprising said first nucleic acid and concomitantly a reduction of the fucosylation of a heterologous polypeptide expressed in such a cell of 90% or more can be achieved. In one embodiment the first nucleic acid preferably comprises a nucleic acid selected from the group of nucleic acids comprising SEQ ID NO: 14 and 15. In another embodiment comprises the first nucleic acid a nucleic acid sequence of SEQ ID NO: 15, or SEQ ID NO: 16.

The first nucleic acid may comprise an additional nucleic acid of a length of 9 residues. Preferably said additional nucleic acid has the sequence of SEQ ID NO: 17.

| | |
|---|---|
| TTCAAGAGA | (SEQ ID NO: 17) |

A nucleic acid according to SEQ ID NO: 17 is denoted as loop of a shRNA. Therefore in one embodiment comprises the first nucleic acid in 5′ to 3′ direction a nucleic acid of SEQ ID NO: 14, 15, or 16, and a nucleic acid of SEQ ID NO: 17. In a preferred embodiment comprises the first nucleic acid in 5′ to 3′ direction a nucleic acid of SEQ ID NO: 14, 15, or 16, directly followed by a nucleic acid of SEQ ID NO: 17, directly followed by a nucleic acid complementary to the complete sequence of SEQ ID NO: 14, 15, or 16. The term “directly followed” as used within this application denotes that two nucleic acid molecules are fused to each other, i.e. the last 3′ terminal nucleotide of the first nucleic acid is directly and covalently linked to the first 5′ terminal nucleotide of the second nucleic acid. The complementary sequence is chosen in a way that it is complementary to the sequence of the nucleic acid directly preceding the nucleic acid of SEQ ID NO: 17.

Plasmid-derived shRNAs are used in the current invention. Cell transformants according to the invention can be obtained with substantially any kind of transfection method known to a person skilled in the art. For example, the plasmid DNA may be introduced into the cells by means of electroporation or microinjection. Alternatively, lipofection reagents such as FuGENE 6 (Roche Diagnostics GmbH), X-tremeGENE (Roche Diagnostics GmbH), nucleofection (AMAXA GmbH, Germany), and Lipofectamine™ (Invitrogen Corp.) may be used. Still alternatively, the vector DNA comprising an expression cassette constituting an shRNA compound may be introduced into the cell by appropriate viral vector systems based on retroviruses, lentiviruses, adenoviruses, or adeno-associated viruses (Singer, O., Proc. Natl. Acad. Sci. 101 (2004) 5313-5314). Preferably electroporation and lipofection are used.

“Heterologous DNA” or “heterologous polypeptide” refers to a DNA molecule or a polypeptide, or a population of DNA molecules, or a population of polypeptides, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing a non-host DNA segment encoding a polypeptide operably linked to a host DNA segment comprising a promoter is considered to be a heterologous DNA molecule. Conversely, a heterologous DNA molecule can comprise an endogenous structural gene operably linked with an exogenous promoter. A polypeptide encoded by a non-host DNA molecule is a “heterologous” polypeptide. “Operably linked” refers to a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader/signal sequence and a polypeptide, contiguous and in reading frame. A polyadenylation site is operably linked to a coding sequence if it is located at the downstream end of the coding sequence such that transcription proceeds through the coding sequence into the polyadenylation sequence. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

The expression “viable” denotes a cell that is capable of propagating, i.e. growing and surviving, when the cell is cultivated in the presence of Lens culinaris agglutinin (LCA) and/or a selection agent. Preferably the cell is cultivated in the presence of LCA. LCA can be added to the cultivation medium at a concentration of from 0.001 mg/ml to 10 mg/ml, preferably of from 0.005 mg/ml to 5 mg/ml, preferably of from 0.01 mg/ml to 1 mg/ml, preferably of from 0.015 mg/ml to 0.5 mg/ml, preferably of from 0.02 mg/ml to 0.4 mg/ml. The concentration of LCA should be at least the concentration at which a mammalian cell not transfected with a nucleic acid according to the invention is not viable. The concentration of LCA is preferably constant in the cultivation. Also preferably the concentration of LCA is increasing in the cultivation. This increasing can be linearly or step wise.

The current invention comprises a method for selecting a mammalian cell, wherein the method comprises the following steps
a) transfecting a mammalian cell with a nucleic acid comprising a first nucleic acid which comprises a part of a nucleic acid encoding a polypeptide that catalyzes an α1,6-glycosidic bond formation between fucose and an asparagine-linked N-acetylglucosamine,
b) cultivating the transfected mammalian cell in the presence of Lens culinaris agglutinin (LCA), and
c) selecting a mammalian cell viable under the conditions of step b).

In one embodiment the mammalian cell is transfected with a nucleic acid comprising a first nucleic acid, which is transcribed to a short hairpin nucleic acid (shRNA) and which comprises a part of a nucleic acid encoding a polypeptide that catalyzes an α1,6-glycosidic bond formation between fucose and an asparagine-linked N-acetylglucosamine. In another embodiment is the mammalian cell transfected with a nucleic acid comprising a first nucleic acid, which is transcribed to a short hairpin nucleic acid (shRNA), which comprises i) a part of a nucleic acid encoding a polypeptide that catalyzes an α1,6-glycosidic bond formation between fucose and an asparagine-linked N-acetylglucosamine, and ii) a nucleic acid of the same length as the nucleic acid of i) and complementary to the complete nucleic acid of i).

In another embodiment the polypeptide that catalyzes an α1,6-glycosidic bond formation between fucose and an asparagine-linked N-acetylglucosamine is α1 ,6-fucosyltransferase.

In one embodiment the first nucleic acid comprises a partial nucleic acid of a complete nucleic acid encoding a polypeptide or protein, whereby the complete nucleic acid is selected from the group of nucleic acids comprising the nucleic acid sequences corresponding to the amino acid sequences of SEQ ID NO: 01 to 11 and the nucleic acid sequences of SEQ ID NO: 12 and 13. In another embodiment the first nucleic acid comprises a nucleic acid selected from the group of nucleic acids comprising the nucleic acid sequences of SEQ ID NO: 14 to 16.

In one embodiment the nucleic acid comprises a second nucleic acid encoding a selection marker. In another embodiment the nucleic acid comprises a third nucleic acid encoding a heterologous polypeptide.

The current invention further comprises a method for selecting a mammalian cell expressing a heterologous polypeptide, wherein the method comprises the following steps
a) transfecting a mammalian cell with a nucleic acid comprising
   i) a first nucleic acid that is transcribed to a short hairpin nucleic acid (shRNA) comprising a part of a nucleic acid encoding a polypeptide that catalyzes an α1,6-glycosidic bond formation between fucose and an asparagine-linked N-acetylglucosamine,
   ii) a second nucleic acid encoding a selection marker,
   iii) a third nucleic acid encoding a heterologous polypeptide,
b) cultivating the transfected mammalian cell in the presence of Lens culinaris agglutinin (LCA), and
c) selecting a mammalian cell viable under the conditions of step b) and thereby selecting a mammalian cell expressing a heterologous polypeptide.

Preferably the method comprises two additional steps a1) and a2) after step a) and before step b),
a1) cultivating said transfected mammalian host cell in the presence of a selection agent,
a2) selecting a transfected mammalian cell viable under the conditions of step a1),
   whereby the transfected mammalian cell selected in step a2) is further cultivated in step b) in the presence of LCA. The selection agent used in step a1) corresponds to the selection marker encoded by the second nucleic acid.

The cultivation of the transfected cells is performed under conditions suitable for the growth of the transfected cell in the absence of LCA and/or a selection agent. These conditions are determined therefore in the absence of LCA or a selection agent, i.e. by cultivating the cells without a selection agent or LCA added to the culture medium. These conditions can easily be determined by a person skilled in the art if not already known by said person.

Preferably the first nucleic acid is comprised in an expression cassette. Preferably said second nucleic acid is comprised in an expression cassette. Preferably said third nucleic acid is comprised in an expression cassette.

The current invention further comprises a nucleic acid comprising
a) a first nucleic acid that is transcribed to a short hairpin nucleic acid (shRNA) comprising a part of a nucleic acid encoding a polypeptide that catalyzes an α1,6-glycosidic bond formation between fucose and an asparagine-linked N-acetylglucosamine,
b) a second nucleic acid encoding a selection marker,
c) a third nucleic acid encoding a heterologous polypeptide.

In one embodiment the first nucleic acid which is transcribed to a short hairpin nucleic acid comprises a nucleic acid selected from the group of nucleic acids of SEQ ID NO: 14, 15, 16. Preferably the first nucleic acid is of SEQ ID NO: 20 or SEQ ID NO: 21. In a further embodiment the heterologous polypeptide is selected from the group of heterologous polypeptides comprising immunoglobulins, immunoglobulin fragments, immunoglobulin conjugates. In another embodiment comprises the first nucleic acid a further nucleic acid which is complementary to the complete nucleic acid of SEQ ID NO: 14, 15, or 16. In this embodiment is the complementary nucleic acid binding without a mismatch to the nucleic acid of SEQ ID NO: 14, 15, or 16 which is comprised in the first nucleic acid. In this embodiment has the complementary nucleic acid the same length as the nucleic acid to which it is complementary.

The cultivation in the presence of LCA according to the method of the invention can be performed in different ways. In one embodiment the cells are cultivated after the transfection in the absence of LCA until a predetermined cell density is obtained and thereafter the cells are cultivated in the presence of LCA. In one embodiment the cells are cultivated after the transfection in the absence of LCA until the cell density has increased by a predetermined factor and thereafter the cells are cultivated in the presence of LCA. Preferably the cell density has increased to 2 times, to 5 times, or to 10 times the cell density at the beginning of the cultivation. In an other embodiment the transfected cells are cultivated after transfection and prior to the cultivation in the presence of LCA in the presence of a selection agent different from LCA, e.g. an antibiotic, if the transfected nucleic acid also comprises a selection marker as second nucleic acid. Preferably said selection marker is the neomycin selection marker. Neomycin is added to the cultivation medium at a concentration of from 50 μg/ml to 1000 μg/ml, preferably of from 100 μg/ml to 800 μg/ml, preferably of from 200 μg/ml to 600 μg/ml. In one embodiment the cells are cultivated for a certain time, i.e. for 3 to 28 days, for 4 to 21 days, preferably for 7 to 14 days, in the presence of neomycin and thereafter for a certain time, i.e. for 3 to 28 days, for 4 to 21 days, preferably for 7 to 14 days, in the presence of LCA.

The expression of the first nucleic acid is preferably mediated by a Pol III promoter, preferably the U6 promoter.

In one embodiment the mammalian cell is transfected with a nucleic acid comprising a nucleic acid encoding a heterologous polypeptide. Therefore the current invention further comprises a method for selecting a mammalian cell expressing a heterologous polypeptide wherein the expressed heterologous polypeptide has a reduced degree of fucose modification, wherein the method comprises the following steps
a) transfecting a mammalian cell with a nucleic acid comprising
   i) nucleic acid that is transcribed to a short hairpin nucleic acid (shRNA) comprising a part of a nucleic acid encoding a polypeptide that catalyzes an α1,6-glycosidic bond formation between fucose and an asparagine-linked N-acetylglucosamine,
   ii) a nucleic acid encoding a heterologous polypeptide,
b) cultivating the transfected mammalian cell in the presence of Lens culinaris agglutinin (LCA), and
c) selecting a mammalian cell viable under the conditions of step b).

Preferably said heterologous polypeptide is an immunoglobulin, or an immunoglobulin fragment, or an immunoglobulin conjugate. Also preferably is said nucleic acid of SEQ ID NO: 20 or SEQ ID NO: 21.

The term “heterologous polypeptide having a reduced degree of fucose modification” and grammatical equivalents thereof denote a heterologous polypeptide, which is expressed in a mammalian host cell transfected with a nucleic acid comprising the first and third nucleic acid according to the invention, and whose fucosylation at the 6-position of an asparagine-linked N-acetylglucosamine is reduced compared to a polypeptide expressed in a mammalian host cell transfected with a nucleic acid comprising the third nucleic acid but not the first nucleic acid according to the invention. Preferably, the ratio of non-fucosylated heterologous polypeptide to fucosylated heterologous polypeptide is 0.15 or less, e.g. 0.12.

In a preferred embodiment the cultivating of the transfected cell is performed in the presence of different concentrations of LCA. LCA is added to the cultivation medium at a concentration of from 0.001 mg/ml to 10 mg/ml, preferably of from 0.005 mg/ml to 5 mg/ml, preferably of from 0.01 mg/ml to 1 mg/ml, preferably of from 0.015 mg/ml to 0.5 mg/ml, preferably of from 0.02 mg/ml to 0.4 mg/ml. In one embodiment the cultivation is performed with a constant concentration of LCA. In a further embodiment the cultivation is performed in the beginning, for a certain time, with a low LCA concentration of from 0.001 mg/ml to 0.1 mg/ml. Afterwards the concentration is increased to a final concentration of from 0.2 mg/ml to 0.5 mg/ml, for a certain time.

The increase of the concentration of the selection agent can be accomplished by a stepwise increase, by a continuous increase, or by a combination of stepwise and continuous increase. If a stepwise increase is performed the concentration can be raised to the final concentration either in multiple steps, e.g. 5 to 10 steps, or in a few steps, e.g. 1 to 3 steps. In case of a continuous increase the concentration may be raised linearly, exponentially, or asymptotically to the final concentration.

In one embodiment the first nucleic acid is selected from the group of nucleic acids of SEQ ID NO: 20 and 21, i.e. has either the nucleic acid sequence of SEQ ID NO: 20, or the nucleic acid sequence of SEQ ID NO: 21.

Selection with a recombinantly expressed cell surface marker can also be used for the isolation of transfectants either alone or in combination with the method of the current invention. It may be used any kind of gene whose expression product is located on the cell surface as a marker for enrichment and selection of transfectants expressing a high level of a shRNA compound. 1-NGFR, a truncated form of the low-affinity nerve growth factor receptor, and thus inactive for signal transduction, is expressed on the cell surface and has proven to be a highly useful marker for cell biological analysis (Philipps, K., et al., Nat. Med. 2 (1996) 1154-1156 and Machl, A. W., et al., Cytometry 29 (1997) 371-374).

The mammalian cell used in the method of the invention is preferably selected from the group comprising hybridoma cells and rodent cells. Also preferably said rodent cell is selected from the group consisting of hamster, mouse, and rat cells.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Annotated vector map of pSilencer2.1_U6neo_antibody_shRNAFuT8

FIG. 2 Annotated vector map of pSilencer2.1_U6neo_antibody_shRNAFuT8_stuffer

FIG. 3 Annotated vector map of pSilencer2.1_U6neo_{—}1-NGFR_shRNAFuT8

FIG. 4 Concentration of the antibody in cell supernatant expressed from different plasmids and selected with neomycin and/or LCA after seven days of cultivation.
Neo: selection with 400 μg/ml neomycin for one week
LCA: selection with 0.05 mg/ml LCA for two weeks followed by selection with 0.5 mg/ml for one week
Neo/LCA: selection with 400 μg/ml neomycin for two weeks followed by selection with 0.5 mg/ml LCA for one week
antibody-vector: pSilencer2.1_U6neo_antibody_shRNAFuT8 antibody-stuffer-vector:
pSilencer2.1_U6neo_antibody_shRNAFuT8_stuffer
X-Axis: 1: antibody-vector, Neo; 2: antibody-vector, Neo/LCA; 3: antibody-stuffer-vector, Neo; 4: antibody-stuffer-vector, Neo/LCA; 5: antibody-stuffer-vector, LCA
Y-Axis: immunoglobulin concentration in μg/ml

FIG. 5 FACS analysis of
a) CHO DG44 cells expressing an antibody but not transfected with pSilencer2.1_U6neo_{—}1-NGFR_shRNAFuT8;
b) CHO DG44 cells expressing an antibody, additionally stably transfected with pSilencer2.1_U6neo_{—}1-NGFR_shRNAFuT8 and selected by neomycin;
c) CHO DG44 cells expressing an antibody, additionally stably transfected with pSilencer2.1_U6neo_{—}1-NGFR_shRNAFuT8, selected by neomycin and subsequently by Lens culinaris agglutinin (LCA).

FIG. 6 Overlay of FIG. 5a to 5c; solid line FIG. 5a), dashed line FIG. 5b), shaded line FIG. 5c).

FIG. 7 Mass spectrum indicating amounts of antibody with different fucosylation isolated from LCA-clone 9.

FIG. 8 Schematic presentation of carbohydrate structures attached to Asn297 or Asn298 of an antibody as deduced from MS-analysis (GlcNAc=N-acetylglucosamine, Man=mannose, Gal=galactose, Fuc=fucose, NeuAc=N-acetyl neuraminic acid)

### EXAMPLES

Example 1 Vector cloning of pSilencer2.1_U6neo_antibody_shRNAFuT8
Example 2 Vector cloning of pSilencer2.1_U6neo_antibody_shRNAFuT8_stuffer
Example 3 Vector cloning of pSilencer2.1_U6neo_{—}1-NGFR_shRNAFuT8
Example 4 Selection and isolation of single CHO DG44 clones with reduced FuT8 expression
Example 5 Selection and isolation of single CHO DG44 clones containing two vectors: pSilencer2.1_U6neo_{—}1-NGFR_shRNAFuT8 and an expression vector of an antibody
Example 6 Selection and isolation of single CHO DG44 clones containing one vector pSilencer2.1_U6neo_antibody_shRNAFuT8 or pSilencer2.1_U6neo_antibody_shRNAFuT8_stuffer
Example 7 One selection method
Example 8 Purification of an antibody from culture supernatant of CHO cells expressing FuT8-shRNA and an antibody
Example 9 FACS-analysis of the cells obtained in Example 6
Example 10 RNA isolation and cDNA synthesis and quantitative RT-PCR
Example 11 Stability of silencing effect and stability of immunoglobulin expression
Example 12 Mass spectrometry analysis of antibody glycostructure

### Example 1

### Vector Cloning of pSilencer2.1_U6neo_antibody_shRNAFuT8

At position 184 of the pSilencer2.1_U6neo vector (Ambion Inc., cat. no. 5764) an XhoI site was introduced by site directed mutagenesis. In order to generate a polylinker containing an AscI and an FseI site, the following oligonucleotides were annealed and ligated between the XhoI/HindIII restriction sites.

| | |
|---|---|
| Polylinker_top | |
| TCGAGGGCGCGCCAGCTCGGGCCGGCCA | (SEQ ID NO: 18) |
| Polylinker_bot | |
| AGCTTGGCCGGCCCGAGCTGGCGCGCCC | (SEQ ID NO: 19) |

As example a genomically organized expression cassette encoding an immunoglobulin binding to the human insulin like growth factor receptor 1 was subsequently cloned between the AscI/FseI restriction sites (for sequences see e.g. WO 2005/005635). In order to generate the FuT8 shRNA, the following oligonucleotides were annealed:

F8shRNA4top

(SEQ ID NO: 20)

GATCCGCCAGAAGGCCCTATTGATCTTCAAGAGAGATCAATAGGGCCTTC
TGGTATTTTTTGGAAA
F8shRNA4bot

(SEQ ID NO: 21)

AGCTTTTCCAAAAAATACCAGAAGGCCCTATTGATCTCTCTTGAAGATCA
ATAGGGCCTTCTGGCG

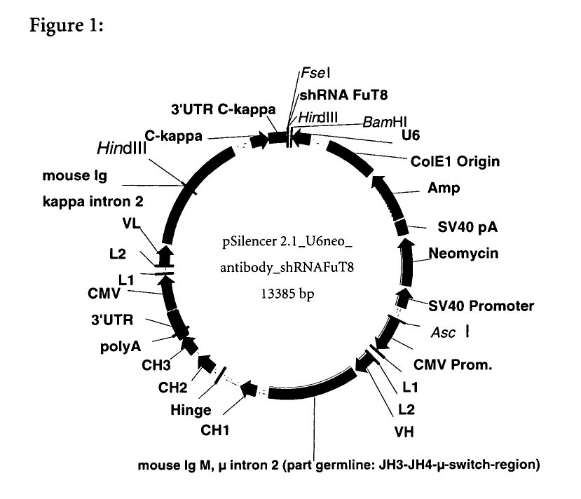

The annealed FuT8 shRNA was ligated into the corresponding vector fragment (BamHI/HindIII digested). The completed vector was called pSilencer2.1_U6neo_antibody_shRNAFuT8. An annotated vector map is shown in FIG. 1.

### Example 2

### Vector Cloning of pSilencer2.1_U6neo_antibody_shRNAFuT8_stuffer

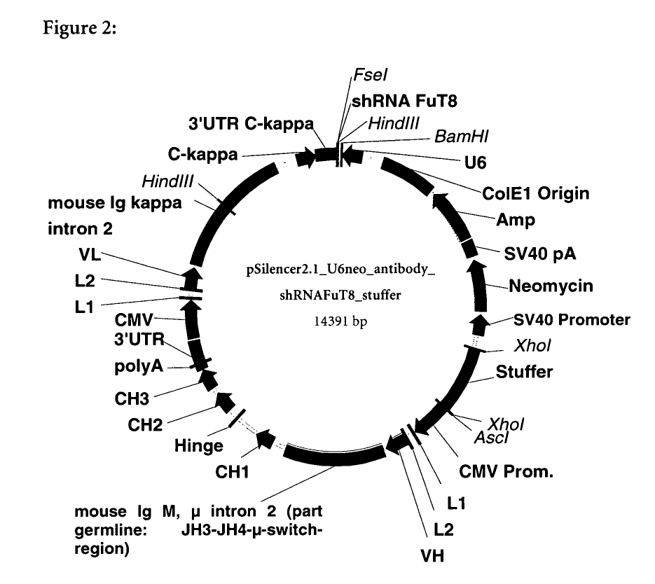

The expression cassettes of the neomycin resistance gene and the expression cassette encoding the antibody in pSilencer2.1_U6neo_antibody_shRNAFuT8 are orientated in different directions. A stuffer sequence was introduced between the promoters of these expression cassettes via the XhoI restriction site. An annotated vector map of the vector pSilencer2.1_U6neo_antibody-shRNAFuT8_stuffer is shown in FIG. 2.

### Example 3

### Vector Cloning of pSilencer2.1_U6neo_{—}1-NGFR_shRNAFuT8

At position 184 of the pSilencer2.1_U6neo vector (Ambion Inc., cat. no. 5764) an XhoI site was introduced by site directed mutagenesis. An 1-NGFR (low affinity nerve growth factor; see e.g. Philipps, K., et al., Nat. Med. 2 (1996) 1154-1156 and Machl, A. W., et al., Cytometry 29 (1997) 371-374) expression cassette was subsequently cloned between the XhoI/HindIII restriction sites. In order to generate the FuT8 shRNA, the following oligonucleotides were annealed:

F8shRNA4top

(SEQ ID NO: 20)

GATCCGCCAGAAGGCCCTATTGATCTTCAAGAGAGATCAATAGGGCCTTC
TGGTATTTTTTGGAAA
F8shRNA4bot

(SEQ ID NO: 21)

AGCTTTTCCAAAAAATACCAGAAGGCCCTATTGATCTCTCTTGAAGATCA
ATAGGGCCTTCTGGCG

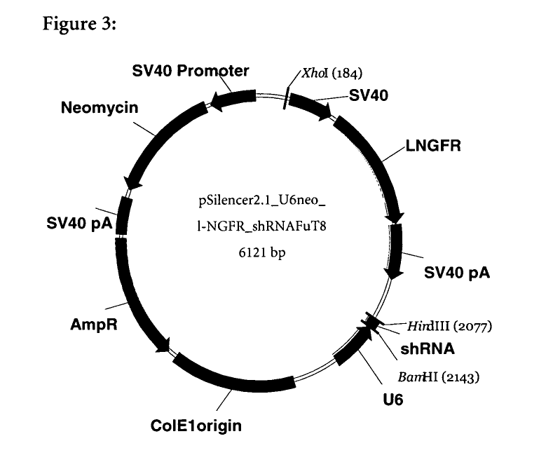

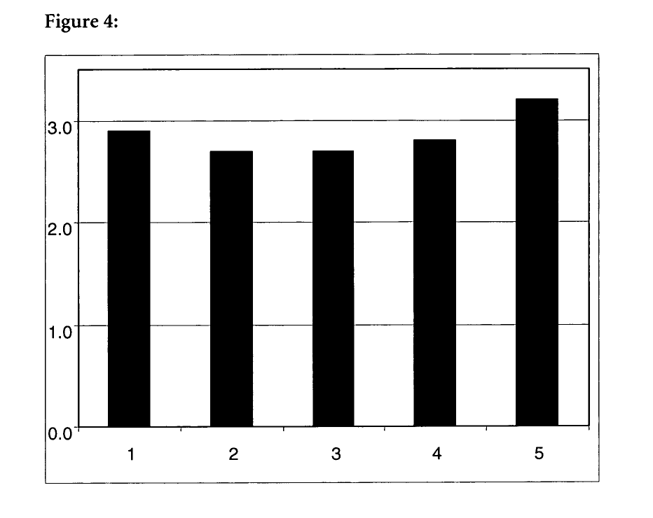

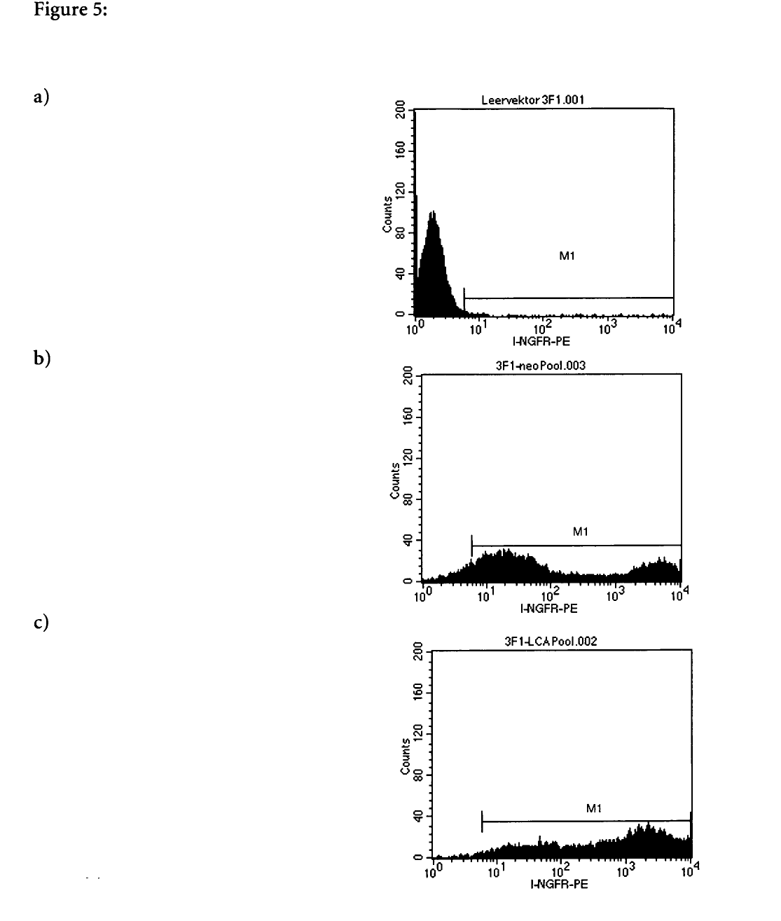

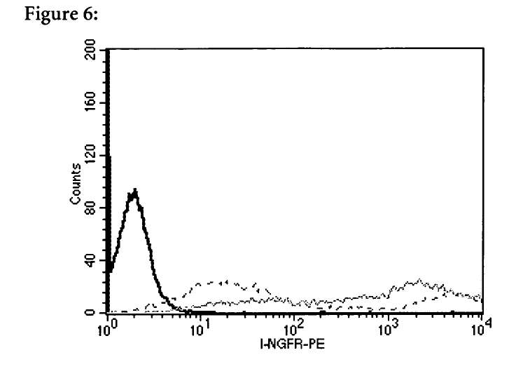

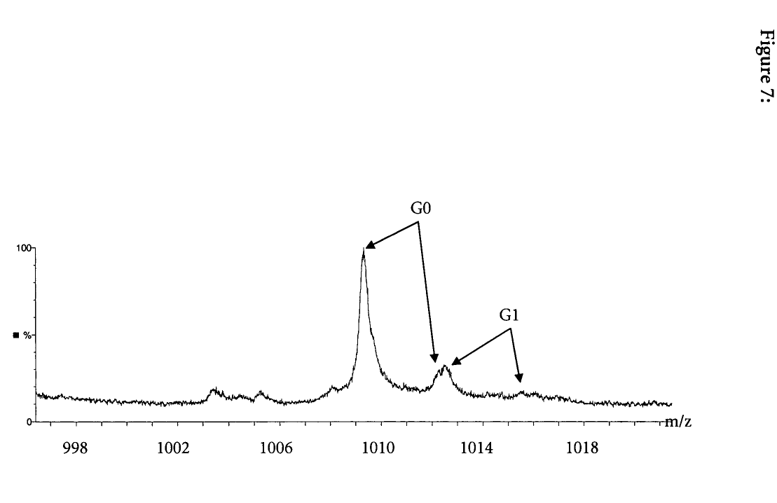

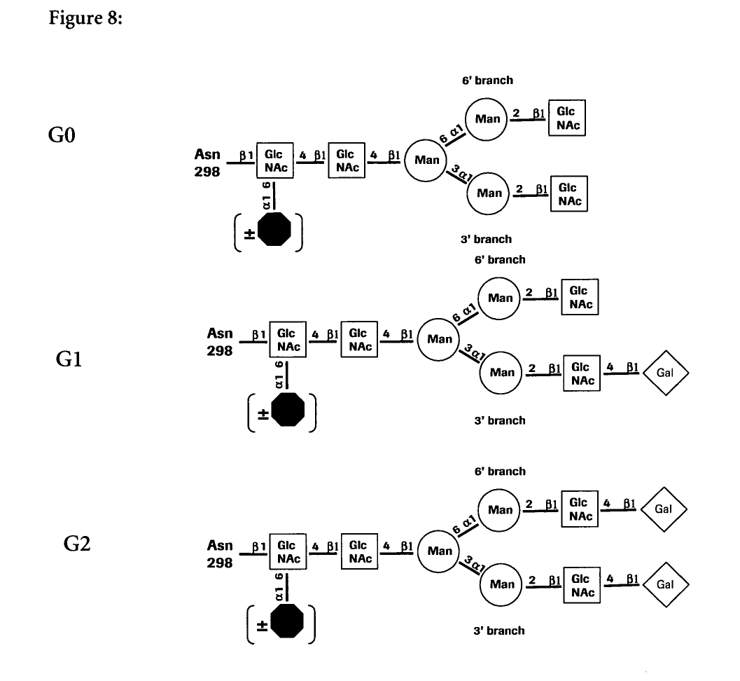

The annealed FuT8 shRNA was ligated into the corresponding vector fragment (BamHI/HindIII digested). The vector is denoted pSilencer2.1_U6neo_{—}1-NGFR_shRNAFuT8. An annotated vector map is shown in FIG. 3.

### Example 4

### Selection and Isolation of Single CHO DG44 Clones with Reduced FuT8 Expression

CHO DG44 cells were transfected with the vector pSilencer2.1_U6neo_{—}1-NGFR_shRNAFuT8 using the FuGENE reagent (Roche Applied Science, Germany) according to the producer's manual. Stably transfected cells were cultured in MEM Alpha Minus Medium (cat. no. 32561; Gibco®, Invitrogen GmbH, Germany) supplemented with 1% (v/v) 200 mM L-glutamine (Gibco), 10% dialyzed gamma irradiated Fetal Bovine Serum (cat. no. 1060-017; Gibco®, Invitrogen GmbH, Germany), and 10 ml HT-Supplement (cat. No. 41065-012; Gibco®, Invitrogen GmbH, Germany). Transfected cells were selected with 400 μg/ml neomycin for two weeks. The neomycin resistant pool was selected afterwards with 0.5 mg/ml LCA (Lens culinaris agglutinin) for an additional week. Single clones were recovered by limited dilution.

The comparison of the 1-NGFR status in cells expressing pSilencer2.1_U6neo_{—}1-NGFR_shRNAFuT8 that were only selected with neomycin with cells that were selected with neomycin and LCA showed this is a very potent agent for selection of recombinant cells.

### Example 5

### Selection and Isolation of a CHO DG44 Pool Containing Two Vectors: pSilencer2.1_U6neo_{—}1-NGFR_shRNAFuT8 and an Expression Vector of an Antibody

CHO DG44 cells were stably transfected with an antibody expression vector. More precisely, at first, an antibody producing CHO DG44 clone was generated using DHFR (dihydrofolatreductase) selection with a vector encoding the mouse DHFR gene (Noe, V., et al., Eur. J. Biochem. 268 (2001) 3163-3173) and a genomically organized nucleic acid of an antibody (see e.g. WO 2005/005635). A single clone was recovered by limited dilution.

The antibody producing CHO DG44 clone was then transfected with the vector pSilencer2.1_U6neo_{—}1-NGFR_shRNAFuT8 using the FuGENE reagent (Roche Diagnostics GmbH, Germany) according to the producer's manual. Stably transfected cells were cultured in MEM Alpha Medium (cat. no. 22561-021; Gibco®, Invitrogen GmbH, Germany) supplemented with 1% (v/v) 200 mM L-glutamine (Gibco) and 10% dialyzed gamma irradiated Fetal Bovine Serum (cat. no. 1060-017; Gibco). Transfected cells were selected with 400 μg/ml neomycin for two weeks. The neomycin resistant pool was selected afterwards with 0.5 mg/ml LCA (Lens culinaris agglutinin) for an additional week.

### Example 6

### Selection and Isolation of a CHO DG44 Pool Containing One Vector pSilencer2.1_U6neo_antibody_shRNAFuT8 or pSilencer2.1_U6neo_antibody_shRNAFuT8_stuffer

CHO DG44 cells were transfected with the vector pSilencer2.1 U6neo_antibody_shRNAFuT8 or pSilencer2.1_U6neo_antibody_shRNAFuT8_stuffer using the FuGENE reagent (Roche Diagnostics GmbH, Germany) according to the producer's manual. The stably transfected cells were cultured in MEM Alpha Minus Medium (cat. no. 32561; Gibco®, Invitrogen GmbH, Germany) supplemented with 1% (v/v) 200 mM L-glutamine (Gibco), 10% dialyzed gamma irradiated Fetal Bovine Serum (cat. no. 1060-017; Gibco®, Invitrogen GmbH, Germany), and 10 ml HT-Supplement (cat. No. 41065-012; Gibco). Transfected cells were selected with 400 μg/ml neomycin for two weeks. The neomycin resistant cells were afterwards selected with 0.5 mg/ml LCA (Lens culinaris agglutinin) for an additional week.

The comparison of the antibody titer in cells expressing pSilencer2.1_U6neo_antibody_shRNAFuT8 that were selected only with neomycin with cells that were selected with neomycin and LCA affirmed that LCA in combination with an shRNAFuT8 expressing vector is a very potent agent for selection of cells expressing recombinant proteins.

### Example 7

### Selection Method

CHO DG44 cells were stably transfected with an antibody expression plasmid and the antibody producing CHO DG44 clone was then transfected with the vector pSilencer2.1_U6neo_{—}1-NGFR_shRNAFuT8 as described in Example 4.

Transfected cells were grown in the presence of a low LCA (Lens culinaris agglutinin) concentration of 0.05 mg/ml for two weeks. The pool resistant to low LCA concentration was grown afterwards at 0.5 mg/ml LCA for an additional week.

Optionally a growth in the presence of 400 μg/ml neomycin for two weeks may precede the LCA selection.

### Example 8

### Purification of Antibody from Culture Supernatant of CHO Cells Expressing FuT8-shRNA and Antibody and Determination of the Immunoglobulin Concentration

About 5-10 ml culture supernatant containing antibody (concentration approximately 5-20 μg/ml) produced by CHO cells also expressing shRNA of FuT8 was incubated with about 100 μl of a suspension of protein A-Sepharose™ CL-4B (30 mg/100 μl; Amersham Pharmacia Biotech AB) at 4° C. over night with inverting of the vial. Subsequently, the sample was centrifuged in an Eppendorf centrifuge 5810R for 15 minutes at about 400×g to sediment the protein A-Sepharose™ to which the immunoglobulin is bound. The culture supernatant was removed completely and the pellet was washed three times each with about 50 μl doubly distilled water. Afterwards the solution was completely removed. About 30 to 50 μl of a 100 mM citrate-buffer, pH 2.8, was added to the pellet, which was subsequently incubated with shaking for 15 minutes at room temperature in order to release the antibody protein bound to the protein A. After incubation, the suspension was centrifuged for 5 minutes at 14,000 rpm in an Eppendorf centrifuge and the resulting supernatant was collected in a second vial (note: supernatant contains the released immunoglobulin). The protein A pellet was washed once again by adding 30 to 50 μl 100 mM citrate buffer, pH 2.8, shaking for about 15 minutes at room temperature and spinning down the protein A-Sepharose™ by centrifugation for 5 minutes at 14,000 rpm. The supernatant was removed carefully and combined with the respective solution of the first release step. The protein A-Sepharose™ pellet was discarded.

The citrate buffer solution containing the immunoglobulin was further on used for the determination of the immunoglobulin concentration. The concentration of the antibody in cell culture supernatants was measured by affinity chromatography on HiTrap™ rProtein Aff (GE healthcare, order no. 17-5079-01). Two hundred fifty microliters of cell culture supernatant were loaded on a 1 ml column which was equilibrated with buffer A (50 mmol/l K₂HPO₄, 300 mmol/l NaCl, pH 7.4). Not bound material was eluted by washing with six column volumes of buffer A followed by six column volumes of buffer B (100 mmol/l sodium acetate, pH 5.0). The bound antibody was eluted with six column volumes buffer C (500 mmol/l sodium acetate, pH 2.5). The protein eluted from the matrix was quantified by UV absorbance and fluorescence spectroscopy. The column was operated at 3 ml/min.

In FIG. 4 five different clones obtained according to Example 6 and Example 7 have been analyzed for the production of the antibody in the culture supernatant. After a cultivation time of 7 days an immunoglobulin concentration of from 2.7 μg/ml to 3.2 μg/ml was obtained in the culture supernatant.

### Example 9

### FACS-Analysis of Cells Obtained in Example 5

Cells as described in Example 5 were seeded in 6-well plates and grown to confluence in a medium according to Example 5. Supernatants were collected and combined in FACS-tubes with the corresponding trypsinized cells. After centrifugation (10 minutes, 1,500 rpm) the supernatants were removed and discarded. Cell pellets were resuspended in a 30 μg/ml monoclonal mouse anti-1-NGFR-antibody solution (Boehringer Mannheim, Germany) and incubated on ice for 30 minutes. After a washing step using 1.5 ml of ice-cold medium, cells were centrifuged for 10 minutes at 1,500 rpm. Supernatants were removed and discarded. The pellets were resuspended in a 20 μg/ml goat (Fab′)₂-anti-Mouse-IgG-(H+L)-PE-antibody solution (Calltag Laboratories; M350004-3) and incubated on ice for 30 minutes. After a washing step using 1.5 ml of ice-cold medium, cells were centrifuged for 10 minutes (1,500 rpm). Supernatants were removed and discarded. The pellets were resuspended in 1 ml of medium and subsequently used for FACS-analysis using a BD-LSR. The results are shown in FIGS. 5 and 6 (designed using the software Cell Quest Pro).

In FIG. 5 the result of the FACS-analyses is displayed. On the X-axis of the graphs the fluorescence intensity is given (increases from left to right), whereas on the Y-axis the detected number of cells exhibiting the corresponding fluorescence intensity is shown.

In FIG. 5a) CHO DG44 transfected with a standard expression vector for the antibody as reference have been analyzed. It can be seen that the detected florescence of the reference cells is located to more than 98% outside the 1-NGFR-PE-fluorescence region designated M1 of 1-NGFR positive cells.

In FIG. 5b) the result of an FACS-analysis of CHO DG44 cells transfected with an expression vector for the antibody and the vector pSilencer2.1_U6neo_{—}1-NGFR_shRNAFuT8 is shown. These cells have been cultivated for one week in the presence of neomycin prior to analysis. The fluorescence intensity detected is proportional to the number of bound goat (Fab′)₂-anti-Mouse-IgG-(H+L)-PE-antibodies to the cell surface and thus proportional to the expression of the shRNA directed against FuT8. It can be seen from FIG. 5b) that after the selection with neomycin more than 90% of the cells exhibit fluorescence inside the preset 1-NGFR-PE-fluorescence region M1 of 1-NGFR positive cells.

In FIG. 5c) the cells that have been analyzed have been cultivated for one week in the presence of neomycin and for an additional week in the presence of LCA (Lens culinaris agglutinin). More than 98% of the analyzed cells exhibit fluorescence intensity inside the preset 1-NGFR-PE-fluorescence region M1 of 1-NGFR positive cells. Additionally, compared to the cells only grown in the presence of neomycin, the fluorescence intensity maximum has been shifted to higher absolute fluorescence intensity.

### Example 10

### RNA Isolation and cDNA Synthesis and Quantitative RT-PCR

Total RNA was isolated using the RNeasy Mini Kit (Qiagen GmbH, Germany) including DNAse digestion. Equal amounts of total RNA (400 ng) were reverse transcribed using the Transcriptor First Strand cDNA Synthesis Kit (Roche Diagnostics GmbH, Germany) with anchored oligo (dT)₁₈ primers.

Samples were analyzed by real-time PCR after cDNA synthesis using the LightCycler FastStart DNA Master SYBR Green I kit (Roche Diagnostics GmbH, Germany). For amplification and detection of FuT8 (Fucosyltransferase 8) and ALAS (5-aminolaevulinate synthase) cDNA, sequence-specific primers were used as follows:

(SEQ ID NO: 22)

| | |
|---|---|
| FuT8 forward: | 5′-GGCGTTGGATTATGCTCATT-3′ |

(SEQ ID NO: 23)

| | |
|---|---|
| FuT8 reverse: | 5′-CCCTGATCAATAGGGCCTTC-3′ |

(SEQ ID NO: 24)

| | |
|---|---|
| ALAS forward: | 5′-CCGATGCTGCTAAGAACACA-3′ |

(SEQ ID NO: 25)

| | |
|---|---|
| ALAS reverse: | 5′-CTTCAGTTCCAGCCCAACTC-3′ |

Amplification was performed under the following conditions: a 10-minutes pre-incubation step at 95° C., followed by 45 cycles of 10 seconds at 95° C., 10 seconds at 52° C., and 8 seconds at 72° C. (temperature ramp 20° C./second). FuT8 cDNA levels were normalized to those of the housekeeping gene ALAS using LightCycler Relative Quantification Software.

Results are shown in the following Table 2. The LCA selected pool was recovered as described in Example 5 (silencer vector=pSilencer2.1_U6neo_{—}1-NGFR_shRNAFuT8). This LCA Pool was further analyzed as describes in Example 9.

TABLE 2
RT-PCR analysis of FuT8 mRNA expression.

| Pool | % FuT8 mRNA |
|---|---|
| CHO wild type cells | 100 (reference) |
| without silencer vector | |
| CHO cells | 7 |
| with LCA selection and with | |
| silencer vector | |
| CHO cells | 32 |
| with neomycin selection and | |
| with silencer vector | |

The CHO wild type cell is the CHO cell in which vectors have been transfected prior to the transfection. The value obtained from the wild-type has been set to 100%. Further results are shown in the following Table 3. LCA clones 1-11 were recovered as described in Example 5. LCA-clone 1 and LCA-clone 9 were used for further analysis in Examples 11 and 12.

TABLE 3
RT-PCR analysis of FuT8 mRNA expression.

| Clone | % FuT8 mRNA |
|---|---|
| CHO wild type cell | 100 (reference) |
| LCA-clone 1 | 2 |
| LCA-clone 2 | 34 |
| LCA-clone 3 | 16 |
| LCA-clone 4 | 23 |
| LCA-clone 5 | 6 |
| LCA-clone 6 | 5 |
| LCA-clone 7 | 15 |
| LCA-clone 8 | 127 |
| LCA-clone 9 | 3 |
| LCA-clone 10 | 23 |
| LCA-clone 11 | 14 |

TABLE 4
RT-PCR analysis of FuT8 mRNA expression (silencer vector =
pSilencer2.1_U6neo_1-NGFR_shRNAFuT8; antibody vector =
pSilencer2.1_U6neo_antibody_shRNAFuT8; antibody-stuffer vector =
pSilencer2.1_U6neo_antibody_shRNAFuT8_stuffer).

| Pool | % FuT8 mRNA |
|---|---|
| CHO DG44, | 100 (reference) |
| without silencer and | |
| without antibody vector | |
| CHO DG44, | 101 |
| with antibody vector, | |
| neomycin selection | |
| CHO DG44, | 12 |
| with antibody vector, | |
| neomycin and LCA selection | |
| CHO DG44, | 101 |
| with antibody-stuffer vector, | |
| neomycin selection | |
| CHO DG44, | 15 |
| with antibody-stuffer vector, | |
| neomycin and LCA selection | |
| CHO DG44, | 7 |
| with antibody-stuffer vector, | |
| LCA selection | |

Pools have been recovered as described in Example 6 and 7. The recovered pools were used for immunoglobulin purification and determination of the immunoglobulin concentration as it is described in Example 8.

### Example 11

### Stability of Silencing Effect and Stability of Immunoglobulin Expression

CHO DG44 cells and LCA-clone 9, both expressing an antibody, were cultured for four weeks without selection pressure, i.e. in the absence of a selective agent. Every week 1×10⁶ cells were plated on a 6 cm diameter culture dish and incubated for 24 hours. Cells were harvested. RNA isolation, cDNA-synthesis, quantitative RT-PCR and data analysis were performed as in Example 10. Results are shown in Table 5.

TABLE 5
FuT8 expression in LCA-clone 9.

| Clone, | |
|---|---|
| week in which cells were harvested | % FuT8 mRNA |
| CHO DG 44 wild type | 100 (reference) |
| LCA-clone 9, week 1 | 8 |
| LCA-clone 9, week 2 | 9 |
| LCA-clone 9, week 3 | 9 |
| LCA-clone 9, week 4 | 9 |

CHO DG44 cells and CHO DG44-clone 1 both expressing an antibody were plated in 6 well plates (1×10⁶ cells, 6 times each). After 72 and 168 hours, supernatants of three wells were harvested and analyzed for immunoglobulin content. Results are shown in Table 6.

TABLE 6
Immunoglobulin expression in LCA-clone 1 and CHO DG44.

| Sample name | Concentration μg/ml | Average | Std. Dev. |
|---|---|---|---|
| LCA-clone 1-I, 7 days | 44.81 | 47.53 | 2.48 |
| LCA-clone 1-II, 7 days | 48.12 | | |
| LCA-clone 1-III, 7 days | 49.67 | | |
| CHO-clone I, 7 days | 44.92 | 46.66 | 1.73 |
| CHO-clone II, 7 days | 46.67 | | |
| CHO-clone III, 7 days | 48.39 | | |
| LCA-clone 1-I, 3 days | 19.34 | 18.86 | 0.50 |
| LCA-clone 1-II, 3 days | 18.88 | | |
| LCA-clone 1-III, 3 days | 18.35 | | |
| CHO-clone I, 3 days | 22.4 | 22.29 | 0.53 |
| CHO-clone II, 3 days | 22.76 | | |
| CHO-clone III, 3 days | 21.72 | | |

### Example 12

### Mass Spectrometry Analysis of Antibody Glycostructure

The relative contents of sugar chain isoforms at Asn297 and/or Asn298, respectively, of the heavy chain of the antibody were determined in glycosylated, intact heavy chains (HC) by mass spectrometry as described in the following:

A) Purification of Antibody from Culture Supernatant of Cells Expressing the Antibody and FuT8-shRNA

About 5-10 ml culture supernatants containing antibody (conc. ˜5-20 μg/ml) produced by cells also expressing shRNA of FuT8 were incubated with about 100 μl suspension of protein A-Sepharose™ CL-4B suspension (30 mg/100 μl; Amersham Pharmacia Biotech AB) at 4° C. over night while inverting the vials. Subsequently, the samples were centrifuged in Eppendorf centrifuge 5810R for 15 min. at about 400×g to sediment the protein A-Sepharose to which the antibody is bound. The culture supernatants were completely removed and the pellets were washed three times with about 50 μl doubly distilled water. After the third wash the solution was completely removed and about 30-50 μl 100 mM citrate-buffer, pH 2.8, was added to the pellets and incubated while shaking for 15 min. at room temperature in order to release the antibody bound to protein A. After incubation, the suspension was centrifuged for 5 min. at 14,000 rpm in an Eppendorf centrifuge and the resulting supernatant was carefully removed. The protein A pellet was washed once by adding again about 30-50 μl of 100 mM citrate buffer, pH 2.8, shaking for about 15 min. at room temperature, and spinning down the protein A-Sepharose by centrifugation for 5 min. at 14,000 rpm in an Eppendorf centrifuge. The supernatant was removed carefully and combined with the respective solution of the first release step. The protein A pellets were discarded.

### B) Analysis of Fc-Glyco Structures by ESI-Mass Spectrometry

The antibody sample (˜60 μl, containing 20-50 μg) obtained in step A) was denatured and reduced into light chain (LC) and glycosylated heavy chain (HC) by adding 100 μl 6 M guanidine-hydrochloride solution and 60 μl of a TCEP-guanidine-solution (1 M tris-(2-carboxyethyl)-phosphine hydrochloride in 6 M guanidine-hydrochloride) to adjust the antibody solution to 3-4 M guanidine-hydrochloride and 250 mM TCEP. The sample was incubated for 1.5 h at 37° C. The reduced and denaturated sample was desalted by G25 gel filtration with 2% formic acid (v/v) and 40% acetonitrile (v/v) as running buffer and was subjected to offline, static ESI-MS analysis with nanospray needles (Proxeon Cat# ES 387) in a Q-Tof2- or a LCT-mass spectrometer instrument from Waters at a resolution of about 10000. The instrument was tuned according to manufacturer's instructions and calibrated with sodium iodine in a mass range from 500-2000 using a first order polynomial fit. Result for LCA-clone 9 is shown in FIG. 7.

During measurement of samples, routinely, 30-40 single scans in a mass range from 700-2000 were recorded and 10-30 single scans were added to yield the final m/z-spectrum used for evaluation.

Identification of the carbohydrate structures bound to the HC and calculation of the relative content of the individual sugar structure isoforms was done from the m/z spectra obtained. The deconvolution tool of the mass lynx software of waters was used to calculate the masses of the individual glycosylated HC-species detected.

The respective carbohydrate structures attached to HC were assigned according by calculating the mass differences between the masses obtained for the individual glycosylated HC-species and the mass for non-glycosylated HC as deduced from the DNA sequence and by comparing these mass differences with theoretical masses of known N-linked glycol structures of antibodies.

For determination of the ratios the oligosaccharide isoforms, the peak heights of the individual, differently glycosylated HC-species were determined from several selected single charge (m/z)-states, which do not overlap with other signals of other molecule species, like LC etc. For determination of the ratios of the oligosaccharide isoforms, the peak heights of G0+Fuc and G0 (see FIG. 8) were determined from selected single charge (m/z)-states. The relative content of sugar structures with reduced fucosylation, was deduced only from the ratio of the peak heights of the HC-species containing the G0-structure+fucose (G0+Fuc; complex, bi-antennary structure lacking terminal galactose residues and carrying core-fucosylation) and the HC-species containing the G0-structure−fucose (G0−Fuc). For this determination the corresponding peaks within the same charge (m/z)-state were used (e.g. peaks of G0+fucose and G0 without fucose of m/z 45). These species were sufficiently resolved from other structures. Quantitative results are shown in Table 7.

TABLE 7
Percentage of fucosylation as determined by mass spectroscopy

| | 100 - amount of |
|---|---|
| Clone | fucosylation [%] |
| LCA-clone 1 | 88 |
| LCA-clone 9 | 83 |

1-34. (canceled)

35. A method for selecting a mammalian cell, comprising
a) transfecting a mammalian cell with a nucleic acid that comprises a first nucleic acid comprising SEQ ID NO: 14, 15, or 16,
b) cultivating the transfected mammalian cell of step a) in the presence of Lens culinaris agglutinin (LCA), and
c) selecting a mammalian cell viable under the conditions of step b), wherein the first nucleic acid is transcribed to a short hairpin nucleic acid and further wherein the first nucleic acid comprises an additional nucleic acid of SEQ ID NO:17.

36. A method for selecting a mammalian cell, comprising
a) transfecting a mammalian cell with a nucleic acid that comprises a first nucleic acid, which is transcribed to a shRNA with the stem of the molecule being due to inverted repeat sequences, which are 19 to 23 nucleotides in length, comprising SEQ ID NO: 14 as stem of a shRNA,
b) cultivating the transfected mammalian cell of step a) in the presence of Lens cularis agglutinin (LCA), and
c) selecting a mammalian cell viable under the conditions of step b), wherein the first nucleic acid comprises an additional nucleic acid of SEQ ID NO:17.

37. The method according to claim 35, characterized in that said first nucleic acid comprises in 5′ to 3′ direction a nucleic acid of SEQ ID NO: 14, 15, or 16, directly followed by a nucleic acid of SEQ ID NO: 17, directly followed by a nucleic acid complementary to the complete sequence of SEQ ID NO: 14, 15, or 16, whereby the sequence of the nucleic acid complementary to the complete sequence of SEQ ID NO: 14, 15, or 16 is chosen in a way that it is complementary to the sequence of the nucleic acid directly preceding the nucleic acid of SEQ ID NO: 17.

38. The method according to claim 36, characterized in that said first nucleic acid comprises in 5′ to 3′ direction a nucleic acid of SEQ ID NO: 14, 15, or 16, directly followed by a nucleic acid of SEQ ID NO: 17, directly followed by a nucleic acid complementary to the complete sequence of SEQ ID NO: 14, 15, or 16, whereby the sequence of the nucleic acid complementary to the complete sequence of SEQ ID NO: 14, 15, or 16 is chosen in a way that it is complementary to the sequence of the nucleic acid directly preceding the nucleic acid of SEQ ID NO: 17.

39. The method of claim 35, wherein said nucleic acid additionally comprises a second nucleic acid encoding a selection marker, and wherein said method comprises after step a) and before step b) the following steps
a1) cultivating the mammalian cell of step a) in the presence of a selection agent,
a2) selecting a mammalian cell viable under the conditions of step a1).

40. The method of claim 36, wherein said nucleic acid additionally comprises a second nucleic acid encoding a selection marker, and wherein said method comprises after step a) and before step b) the following steps
a1) cultivating the mammalian cell of step a) in the presence of a selection agent,
a2) selecting a mammalian cell viable under the conditions of step a1).

41. The method of claim 35, wherein said nucleic acid comprises a third nucleic acid encoding a heterologous polypeptide selected from the group consisting of immunoglobulins, immunoglobulin fragments, or immunoglobulin conjugates.

42. The method of claim 36, wherein said nucleic acid comprises a third nucleic acid encoding a heterologous polypeptide selected from the group consisting of immunoglobulins, immunoglobulin fragments, or immunoglobulin conjugates.

43. The method of claim 35, wherein said mammalian cell is selected from the group consisting of a CHO cell, a BHK cell, a HEK cell, a PER.C6® cell, a Sp2/0 cell, and a NS0 cell.

44. The method of claim 35, wherein said cultivating in the presence of LCA is in the presence of an increasing concentration of LCA, wherein the LCA concentration ranges from 0.015 mg/ml to 0.5 mg/ml.

45. The method according to claim 44 characterized in that said increasing is linear or stepwise.

46. The method of claim 44 characterized in that said cultivating of the transfected mammalian cells in step b) is in the absence of LCA until a predetermined cell density is obtained and thereafter the cells are cultivated in the presence of LCA.

47. The method according to claim 39, characterized in that the transfected mammalian cells in step b) are cultivated after transfection and prior to the cultivation in the presence of LCA in the presence of a selection agent different from LCA, wherein said selection agent is neomycin.

48. A method for selecting a mammalian cell, characterized in that the method comprises the following steps
a) transfecting a mammalian cell with a nucleic acid comprising a first nucleic acid of SEQ ID NO: 20 or SEQ ID NO: 21
b) cultivating the transfected mammalian cell in the presence of Lens culinaris agglutinin (LCA), and
c) selecting a mammalian cell viable under the conditions of step b).

49. A method for selecting a mammalian cell expressing a heterologous polypeptide wherein the expressed heterologous polypeptide has a reduced degree of fucose modification, wherein the method comprises the following steps
a) transfecting a mammalian cell with a nucleic acid comprising
i) a nucleic acid that is transcribed to a short hairpin nucleic acid (shRNA) of SEQ ID NO:20 or SEQ ID NO: 21,
ii) a nucleic acid encoding a heterologous polypeptide,
b) cultivating the transfected mammalian cell in the presence of Lens culinaris agglutinin (LCA), and
c) selecting a mammalian cell viable under the conditions of step b) as mammalian cell expressing a heterologous polypeptide.

50. A nucleic acid comprising
a) a first nucleic acid, selected from the group of nucleic acids of SEQ ID NO: 20 or 21, which is transcribed to a short hairpin nucleic acid comprising a nucleic acid selected from the nucleic acids of SEQ ID NO: 15 or 16,
b) a second nucleic acid encoding a selection marker,
c) a third nucleic acid encoding a heterologous polypeptide selected from the group of heterologous polypeptides comprising immunoglobulins, immunoglobulin fragments, immunoglobulin conjugates.

51. A nucleic acid comprising
a) a first nucleic acid, selected from the group of nucleic acids of SEQ ID NO: 20 or 21, which is transcribed to a short hairpin nucleic acid with the stem of the molecule being due to inverted repeat sequences, which are 19 to 23 nucleotides in length, comprising a nucleic acid selected from the nucleic acids of SEQ ID NO:14 as stem of a shRNA,
b) a second nucleic acid encoding a selection marker,
c) a third nucleic acid encoding a heterologous polypeptide selected from the group of heterologous polypeptides comprising immunoglobulins, immunoglobulin fragments, immunoglobulin conjugates.
